(19) United States
(12) Patent Application Publication
(10) Pub. No.: US 2019/0291112 A1
(43) Pub. Date: Sep. 26, 2019

(54) DROPLET MICROFLUIDICS FOR DRUG SCREENING

(71) Applicant: University of Macau, Macau (CN)

(72) Inventors: Ada Hang-Heng WONG, Macau (CN); Chuxia DENG, Macau (CN); Yanwei JIA, Macau (CN); Haoran LI, Macau (CN); Pui-In MAK, Macau (CN); Rui Paulo da Silva MARTINS, Macau (CN); Yan LIU, Macau (CN); Chi Man VONG, Macau (CN); Hang Cheong WONG, Macau (CN); Pak Kin WONG, Macau (CN); Haitao WANG, Macau (CN); Heng SUN, Macau (CN)

(21) Appl. No.: 16/360,186

(22) Filed: Mar. 21, 2019

Related U.S. Application Data
(60) Provisional application No. 62/645,816, filed on Mar. 21, 2018.

(51) Int. Cl. B01L 3/00

(52) U.S. Cl. CPC B01L 3/502784; B01L 3/502761; G01N 33/57484; B01L 2300/0867; B01L 2200/027; B01L 2300/0864

(57) ABSTRACT

Provided is a microfluidic chip for generating a plurality of droplets comprising plural droplet-forming units serially connected together, an inlet for receiving the loading fluid and providing the loading fluid to the plural droplet-forming units, and an outlet for discharging the loading fluid remained after passing through the plural droplet-forming units. Each of the individual droplet-forming unit include an inflow channel, a neck channel, a droplet-forming well and a bypass channel therearound, a restricted flow port element, and an outflow channel, the arrangement of which allows the microfluidic chip to form robust and stable droplets for reliable and flexible drug screening assays using a small sample input size.

### CROSS-REFERENCE TO RELATED APPLICATIONS

The present application claims priority to and the benefit of U.S. Provisional Patent Application Ser. No. 62/645,816, entitled DRUG SCREENING OF CANCER CELL LINES AND HUMAN PRIMARY TUMORS USING DROPLET MICROFLUIDICS, which was filed on Mar. 21, 2018, and is hereby incorporated by reference in its entity.

### FIELD OF THE INVENTION

The present disclosure generally relates to a microfluidic system for generating droplets and methods for making and using the same, and in particular, to a microfluidic system and methods useful for, e.g., drug screening of cells, especially cancer cells, and especially primary tumor cells.

### BACKGROUND OF THE INVENTION

Cancer is one of the most lethal diseases, which threatens millions of people worldwide, accounting for approximately 13% of all deaths globally. Although all clinically approved drugs and drug combinations have been tested in vitro using cultured cells, in vivo using animal models and in clinical trials, there is no guarantee that a particular treatment will successfully treat a patient's cancer, due to insufficient knowledge of cancer etiology, diversity of cancer types and properties.

Understanding the heterogeneous drug responses from individual cancer cells within a population of cancer cells can be critical to understanding cancer etiology and cancer diversity. However, most of the current in vitro drug screening platforms provide drug responses from a bulk population of cancer cells, and tend to overlook the heterogeneity of drug responses within the bulk population of cells. Hence, there is a need to develop a drug screening platform that provides drug response from a bulk population of cancer cells, as well as from individual cells within the bulk population of cancer cells.

In vivo drug screening using animal models is generally more reliable yet more expensive than in vitro drug screening using cultured cells. Most primary tumors contain multiple subclones and have genetic heterogeneity. However, the majority of cancer cell lines are propagated through hundreds of passages and as a result a single clone dominates the culture and the genetic heterogeneity of the primary tumor is lost. Primary tumor cells, directly obtained from an animal tumor tissue, closely resemble the parental tumor tissue and have similar biological responses to an in vivo situation. Thus, in vitro drug screening using primary tumor cells directly obtained from tumor tissue could better predict treatment outcome.

However, the major hurdle to drug screening on primary tumor cells is low sample input. For example, while leukemia patients provide over 10 million cancer cells from 2 ml of patient blood, mammary tumors of 2×2 cm can only result in less than 1 million cells in sum after dissociation.

Hence, there is need to develop a reliable and inexpensive drug screening method that requires low sample input.

### SUMMARY OF THE INVENTION

Provided herein is a microfluidic chip that enables, e.g. quick sample loading, robust droplet formation, and/or automatic droplet investigation with a small sample input size. The sample includes but is not limited to cancer cells, such as cancer cell lines, primary tumor cells, secondary tumor cells, cancer stem cells, or circulating tumor cells. Other cell types for other disease models, e.g. metabolic diseases, respiratory diseases, and infectious diseases, are also within the contemplation of the present disclosure.

In a first aspect, a microfluidic system of the present disclosure is capable of loading sample more quickly and requires as short as 5 minutes even with manual loading, whereas conventional methods usually require 1-1.5 hours with manual loading and still require 10-30 min even with automatic loading. The quick sample loading method of the microfluidic system of the present disclosure allows high throughput screening and is beneficial for special samples that can only survive for a short period of time, such as primary tumor cells.

In a second aspect, a microfluidic system of the present disclosure is capable of generating robust droplets and preventing droplet escape during overnight incubation at 37° C. A robust droplet refers to a droplet with minimal loss of sample, no droplet coalescence, e.g. merging of two or more droplets, and/or no cross-contamination between droplets, e.g. mixing of the sample from one droplet with the sample from the other droplet, which are the prerequisites for reliable drug screening of the samples enclosed within the droplets. In existing droplet microfluidic systems, the formed droplets tend to escape from the droplet-forming wells during overnight incubation at 37° C., which causes undesired loss of sample and alteration of screening conditions that can ultimately affect drug screen outcomes. Therefore, the special configuration of the microfluidic system of the present disclosure significantly improves the reliability of the drug screening.

In a third aspect, a microfluidic system of the present disclosure is capable of filling the droplet-forming well fully to maximize channel space usage and save cost.

In a fourth aspect, a drug screening method of the present disclosure is capable of providing efficient evaluation of drug susceptibility of cancers with as few as 16,000 cells obtained from primary cancer sample obtained from a patient for each treatment condition within 24 h. Moreover, the sample input size can be potentially reduced to 100 cells per drug dose based on the configuration of the microfluidic system of the present disclosure. In addition, the cost of the drug screening method of the present disclosure is as low as HKD 0.20 per chip, making it pragmatically affordable for all cancer patients.

In certain embodiments, the present disclosure relates to microfluidic chip 100 for generating a plurality of droplets from a loading fluid, comprising at least one droplet-forming channel 200, each of the at least one droplet-forming channel 200 comprising: plural droplet-forming units serially connected together; an inlet 201 for receiving the loading fluid and providing the loading fluid to the plural droplet-forming units; and an outlet 202 for discharging the loading fluid remained after passing through the plural droplet-forming units; wherein: an individual droplet-forming unit 209 comprising an inflow channel 203, a neck channel 204, a droplet-forming well 205, a restricted flow port element 206, and an outflow channel 207 all of which are sequentially arranged along a flow direction of the loading fluid; the inflow channel 203 is configured to accept the loading fluid and is in fluid communication with the neck channel 204, the neck channel 204 is in fluid communication with the droplet-forming well 205 for delivering a first portion of the loading fluid from the inflow channel 203 to the droplet-forming well 205, and is configured to have a cross-sectional width that is smaller than a cross-sectional width of the droplet-forming well 205 to prevent droplet escape from the droplet-forming well 205; the restricted flow port element 206 is configured to generate a restricted flow to facilitate droplet formation in the droplet-forming well 205; and wherein: the individual droplet-forming unit 209 further comprises a bypass channel 208; the bypass channel 208 is located around the droplet-forming well 205, and is configured to deliver a second portion of the loading fluid from the inflow channel 203 to the outflow channel 207.

in certain embodiments, the neck channel 204 and the bypass channel 208 have a cross-sectional width ratio of the bypass channel to the neck channel, the cross-sectional width ratio being selected such that the first portion of the loading fluid fills the droplet-forming well 205 before the second portion of the loading fluid fills the bypass channel 208.

In certain embodiments, the cross-sectional width ratio of the bypass channel to the neck channel is approximately 0.2 to approximately 1.0.

In certain embodiments, the cross-sectional width ratio of the bypass channel to the neck channel is approximately 075.

In certain embodiments, the neck channel 204 has a cross-sectional width of approximately 50-150 μm.

In certain embodiments, the droplet-forming well 205 has a cross-sectional width of approximately 100-500 μm.

In certain embodiments, the restricted flow port element 206 is a restriction channel having a cross-sectional width of approximately 5-20 μm.

The present disclosure also relates to a mold comprising complementary features to a microfluidic chip 100, the microfluidic chip 100 comprising: at least one droplet-forming channel 200, each of the at least one droplet-forming channel 200 comprising: plural droplet-forming units serially connected together; an inlet 201 for receiving the loading fluid and providing the loading fluid to the plural droplet-forming units; and an outlet 202 for discharging the loading fluid remained after passing through the plural droplet-forming units; wherein: an individual droplet-forming unit 209 comprising an inflow channel 203, a neck channel 204, a droplet-forming well 205, a restricted flow port element 206, and an outflow channel 207 all of which are sequentially arranged along a flow direction of the loading fluid; the inflow channel 203 is configured to accept the loading fluid and is in fluid communication with the neck channel 204, the neck channel 204 is in fluid communication with the droplet-forming well 205 for delivering a first portion of the loading fluid from the inflow channel 203 to the droplet-forming well 205, and is configured to have a cross-sectional width that is smaller than a cross-sectional width of the droplet-forming well 205 to prevent droplet escape from the droplet-forming well 205; the restricted flow port element 206 is configured to generate a restricted flow to facilitate droplet formation in the droplet-forming well 205; and wherein: the individual droplet-forming unit 209 further comprises a bypass channel 208; the bypass channel 208 is located around the droplet-forming well 205, and is configured to deliver a second portion of the loading fluid from the inflow channel 203 to the outflow channel 207.

In certain embodiments, the material for the mold is selected from the group consisting of crystalline silicon, amorphous silicon, glass, quartz, and metals.

In certain embodiments, the neck channel and the bypass channel of the mold have a cross-sectional width ratio of the bypass channel to the neck channel, the cross-sectional width ratio being selected such that the first portion of the loading fluid fills the droplet-forming well before the second portion of the loading fluid fills the bypass channel.

In certain embodiments, the cross-sectional width ratio of the bypass channel to the neck channel of the mold is approximately 0.75.

The present disclosure also relates to a method for drug screening, wherein the method comprising steps of:
a) providing the microfluidic chip 100;
b) flushing the droplet-forming channel 200 with a carrier fluid from the outlet 202 to the inlet 201;
infusing a loading fluid comprising of a sample fluid and a carrier fluid in distinct layers separated by an interface from the inlet 201 into the droplet-forming channel 200 to form droplets comprising the sample fluid;
d) sealing the inlet 201 and the outlet 202; and
e) imaging the droplets comprising the sample fluid.

In certain embodiments, the carrier fluid comprises an oil and a surfactant.

in certain embodiments, the carrier fluid is a perfluorinated trialkyl amine oil supplemented with approximately 1-5% fluorosurfactant.

in certain embodiments, the sample fluid comprises cells, a drug, a cell culture medium, an additive, a dead cell indicator, and/or a metabolic indicator.

In certain embodiments, the cells are cancer cells selected from the group consisting of cancer cell lines, primary tumor cells, secondary tumor cells, cancer stem cells, and circulating tumor cells.

In certain embodiments, the cell culture medium comprises fetal bovine serum at a concentration of 1%-20% (v/v).

In certain embodiments, the dead cell indicator is selected from the group consisting of ethidium homodimer 1, Alamar Blue, SYTOX Green nucleic acid stain, and propidium iodide; and the metabolic indicator is selected from the group consisting of Calcein AM, C12-resazurin, SYTO 10 dye, and SYBR 14 nucleic acid stain.

In certain embodiments, the methyl cellulose has a percentage of 0.5%-3% (m/v) in the sample fluid.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Other aspects and advantages of the invention will be apparent to those skilled in the art from a review of the ensuing description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects and features of the present disclosure will become apparent from the following description of the various embodiments described herein, when taken in conjunction with the accompanying drawings, in which:

FIG. 1 illustrates a plan view of a microfluidic chip according to certain embodiments of the present disclosure.

FIG. 2 illustrates a plan view of a droplet-forming channel of the microfluidic chip and an enlarged plan view of a droplet-forming unit within the channel according to certain embodiments of the present disclosure.

FIG. 3A illustrates the correlation between observed volume (y-axis) and preset volume (x-axis), wherein observed volume was calculated by multiplying the number of occupied wells (at 0.5 increments) by the theoretical well volume (length×width×height) of each well, and wherein preset volume referred to the volume set on syringe pump according to certain embodiments of the present disclosure; error bars denoted standard deviation of mean observed volumes obtained from all replicates for each preset volume.

FIG. 3B illustrates chip occupancy (y-axis) plotted against preset volume (x-axis), wherein occupied wells was the number of occupied wells and preset volume referred to the volume set on syringe pump according to certain embodiments of the present disclosure; error bars denoted standard deviation of the observed occupied wells in all replicates for each preset volume.

FIG. 4A illustrates the sample loading workflow according to certain embodiments of the present disclosure.

FIG. 4B illustrates sample loading and droplet formation according to certain embodiments of the present disclosure.

FIG. 4C illustrates the droplet filling with different cross-sectional width ratios of the bypass channel to the neck channel.

FIG. 5A illustrates a drug screening assay according to certain embodiments of the present disclosure.

FIG. 5B illustrates the image processing workflow according to certain embodiments of the present disclosure.

FIG. 5C shows cells extracted by the image processing workflow according to certain embodiments of the present disclosure. Live cells were marked in blue and dead cells were marked in red.

FIGS. 6A-6D illustrate the optimization of oil phase (carrier fluid) and aqueous phase (sample fluid) of the microfluidic chip according to certain embodiments of the present disclosure.

FIG. 6E illustrates a table for the properties of the drugs and/or dyes used according to certain embodiments of the present disclosure.

FIG. 6F illustrates adherent cells within a droplet with and without the addition of 1% methyl cellulose according to certain embodiments of the present disclosure.

FIG. 6G illustrates the proliferation of Jurkat cells and MDA-MB-231 cells with and without the addition of methyl cellulose according to certain embodiments of the present disclosure.

FIGS. 7A-7D show the frequency plots of the total number of cells observed in each well according to certain embodiments of the present disclosure.

FIGS. 8A-8H show the cell viability of jurkat cells and MDA-MB-231 cells against the log of different drug concentrations according to certain embodiments of the present disclosure; error bars denoted standard deviation of mean cell viability obtained from all replicates in parallel experiments in plate reader assays, whereas for chip assay, error bars denoted standard deviation of mean cell viability obtained from two independent experiments.

FIGS. 9A and 9B show the drug susceptibility of the primary tumor cell NAS1604 and the derived cancer cell line NAS1604C amplified therefrom according to certain embodiments of the present disclosure.

FIG. 9C shows the morphology of the primary tumor cell NAS1604 (left) and the derived cell line NAS1604C amplified therefrom (right).

FIG. 10A shows the drug list according to certain embodiments of the present disclosure.

FIG. 10B shows statistical analysis of drug response of NAS1608 human primary nasopharyngeal tumor sample towards bortezomib and cisplatin according to certain embodiments of the present disclosure.

FIG. 10C shows Tukey's HSD test results according to certain embodiments of the present disclosure.

FIG. 11A illustrates the correlation between the average number of cells per well (x-axis) and the concentration of cells before loading on chip (y-axis).

FIG. 11B illustrates the Poisson distribution of cells per droplet.

FIGS. 12A and 12B illustrate that Ethidium homodimer 1 emits strong red fluorescence in drug treated cells (B), whereas the fluorescence is weaker in DMSO) treated cells (A). Scale bar: 100 μm.

FIGS. 13A-13F show the cell viability of Jurkat cells and MDA-MB-231 cells against the log of different drug concentrations according to certain embodiments of the present disclosure; error bars denoted standard deviation of mean cell viability obtained from all replicates in parallel experiments, except for the chip assay of cisplatin-treated MDA-MB-231 cells (F) where error bars denoted standard deviation of mean cell viability Obtained from all droplets of two independent experiments.

FIGS. 14A-14C show the cell viability of seven primary nasopharyngeal tumors screened against two drugs and mock treatment control with DMSO according to certain embodiments of the present disclosure.

FIGS. 15A-15C show the cell viability of one human nasopharyngeal cancer sample NAS1608 against two drugs and mock treatment control with DMSO according to certain embodiments of the present disclosure.

### DETAILED DESCRIPTION OF THE INVENTION

For the purposes of the present disclosure, cancer cells are described in connection with the microfluidic system and the methods using thereof described herein only as exemplary embodiments. It should be appreciated that the uses of the system and methods are not limited to cancer cells, but also other cell types for different disease models, or other biological samples, e.g. bacteria.

Additionally, to assist in the description of the structural configuration, words such as length, width, height, depth, upper, lower, top, bottom, transverse, longitudinal, horizontal and the like are used. Unless their contextual usage indicates otherwise, these words are to be understood herein as having no structural, functional or operational significance and as merely reflecting the arbitrarily chosen orientation.

The term “channel” or “well” as used herein is to be interpreted in a broad sense. Thus, it is not intended to be restricted to elongated configurations where the transverse dimension or longitudinal dimension greatly exceeds the diameter or cross-sectional dimension. Rather, such term is meant to comprise cavities or tunnels of any desired shape or configuration through which fluids, such as liquids and gases, may be directed. Such a fluid cavity may, for example, comprise a flow-through cell where fluid is to be continually passed or, alternatively, a chamber for holding a specified, discrete amount of fluid for a specified amount of time. “Channels” or “wells” may be filled with or may contain internal structures comprising, for example, valves, filters, or equivalent components and materials. A microfluidic channel can have a cross-sectional dimension in the range between about 1.0 μm and about 500 μm, between about 25 μm and about 200 μm or between about 50 μm and about 150 μm.

The term “transverse dimension” as used herein refers to the dimension of a plane that is parallel to the plane defined by the top or bottom surface of a channel or well and is parallel to the flow direction. The term “longitudinal dimension” as used herein refers to the dimension of a plane that is perpendicular to the plane defined by the top or bottom surface of a channel or well and is parallel to the flow direction. The term “cross-sectional dimension” as used herein refers to the dimension of a plane that is perpendicular to both the transverse plane and the longitudinal plane, and is also perpendicular to the flow direction.

The term “cross-sectional width” or “width” as used herein refers to the dimension that is perpendicular to the longitudinal dimension while parallel to the transverse dimension. The term “length” as used herein refers to the dimension that is parallel to the transverse dimension and longitudinal dimension while perpendicular to the cross-sectional dimension. The term “height” or “depth” as used herein refers to the dimension that is perpendicular to the transverse dimension while parallel to the longitudinal dimension and the cross-sectional dimension.

The term “microfluidic” as used herein is to be understood, without any restriction thereto, to refer to structures or devices through which fluid, such as liquids and gases, is capable of being passed or directed, wherein one or more of the dimensions is less than about 500 μm.

The term “in fluid communication” throughout the present disclosure, unless the context indicates otherwise, does not indicate a fluid must flow from one of the two components in fluid communication directly to the other. There can be one or more other components, such as devices, valves, ports, ducts, tubings, etc. between the two components.

As used herein the term “and/or” includes any and all combinations of one or more of the associated listed items.

The term “serially connected” used herein is not limited to the case where subjects, e.g. droplet-forming units, are directly connected, and thus the term may refer to the case where any other members are disposed between the subjects.

The term “sequentially arranged” as used herein refers to, for example, “A and B are sequentially arranged” representing the elements A and B arranged in the order described above, and the other elements, e.g. C, may be interposed between A and B, for example, A, C, and B may be provided in the order described above.

The term “element” as used herein is intended to include meanings of other like-terms such as “component” and so forth.

The term “complementary features” as used herein refers to a set of features that are complementary to another set of features so that when the two sets of features are merged together there is minimal space left therebetween, :For example, a set of concave features are complementary features to a set of convex features of the same dimensions with the set of concave features.

The term “sample input size” as used herein refers to the volume of sample fluid and/or the total number of cells within a sample fluid.

As used herein, the term “prevent” or “preventing” refers to any method to partially or completely preclude, avert, obviate, forestall, stop, hinder or delay the consequence or phenomenon following the term “prevent” or “preventing” from happening. The term “prevent” or “preventing” does not mean that the method is necessarily absolute, but rather effective for providing some degree of prevention or amelioration of consequence or phenomenon following the term “prevent” or “preventing”.

### Structural Configuration of the Microfluidic Chip

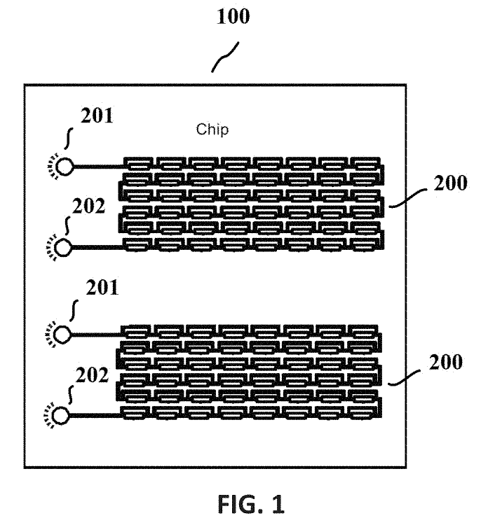

FIG. 1 illustrates a plan view of a microfluidic chip 100 according to certain embodiments of the present disclosure. The microfluidic chip 100 can be used as a lab-on-a-chip (LOC). The microfluidic chip 100 comprises at least one droplet-forming channels 200 arranged on the same plane. In certain embodiments, the droplet-forming channel 200 has a transverse dimension of about 3.0-5.0 mm×5.0-15 mm in the x-y plane, but the dimension can be different according to practical needs, such as the capacity of the droplet-forming channel 200. Therefore, any other reasonable transverse dimensions of the droplet-forming channel 200 are also within the contemplation of the present disclosure. In certain embodiments, the microfluidic chip 100 has 2, 3, 4, 5, 7 8, or more droplet-forming channels 200. Larger quantities numbers of the droplet-forming channels 200 are also within the contemplation of the present disclosure. In certain embodiments, the microfluidic chip 100 has 2 droplet-forming channels 200. The number of droplet-forming channels 200 that can be accommodated on a microfluidic chip 100 can be different according to practical needs, such as the number of experimental conditions to be screened. Therefore, any other number of the droplet-forming channels 200 arranged on one microfluidic chip 100 are also within the contemplation of the present disclosure. In certain embodiments, the microfluidic chip 100 has a transverse dimension of about 1.0-4.0 cm×1.0-4.0 cm in x-y plane, but the dimension can be different according to practical needs and the number of droplet-forming channels 200 to be accommodated thereon. Therefore, any other transverse dimensions of the microfluidic chip 100 are also within the contemplation of the present disclosure.

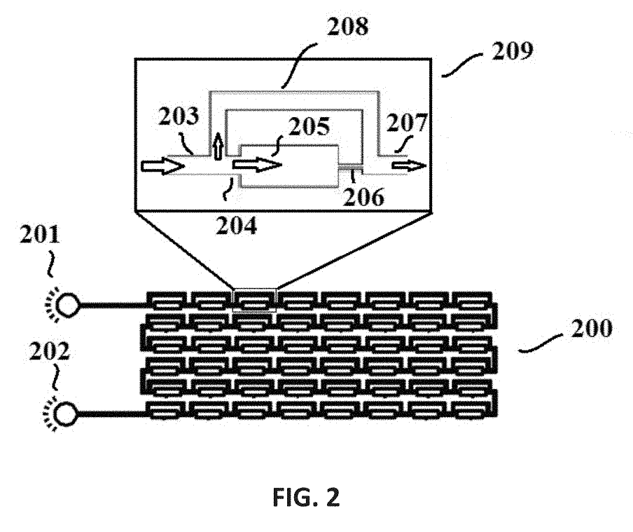

FIG. 2 illustrates a plan view of one droplet-forming channel 200. The droplet-forming channel 200 comprises plural droplet-forming units that are serially connected together, an inlet 201 for receiving a loading fluid and providing the loading fluid to the plural droplet-forming unit, and an outlet 202 for discharging the loading fluid remained after passing through the plural droplet-forming units. In certain embodiments, the number of the droplet-fanning unit 209 within a droplet-forming channel 200 is 10-100, 20-90, 30-80, 40-70, or 50-60. Other numbers of the droplet-forming unit 209 are also within the contemplation of the present disclosure. In certain embodiments, the number of the droplet-forming unit 209 within a droplet-forming channel 200 is 48. The number of the droplet-forming unit 209 within a droplet-forming channel 200 can be different according to practical needs, such as the number of experimental conditions to be screened or the size of the loading fluid. Therefore, any other number of the droplet-forming unit 209 within a droplet-forming channel 200 are also within the contemplation of the present disclosure. In certain embodiments, the plural droplet-forming units are arranged to form a rectangular shape. It should be appreciated that the plural droplet-forming units can be arranged in any desired shape, e.g. a circle, a square, an oval, a triangle, a parallelogram, a heptagon, or an octagon, according to practical needs so as to maximize the space usage of a microfluidic chip 100.

FIG. 2 also illustrates an enlarged plan view of an individual droplet-forming unit 209. The individual droplet-forming unit 209 comprises an inflow channel 203, a neck channel 204, a droplet-forming well 205, a restricted flow port element 206, and an outflow channel 207 all of which are sequentially arranged along a flow direction of the loading fluid passing through the inflow channel 203, the neck channel 204, the droplet-forming well 205, the restricted flow port element 206, and the outflow channel 207. The flow direction of the loading fluid passing through the above-mentioned components is illustrated by the horizontal arrows in FIG. 2 according to certain embodiments. The thickness of the arrows qualitatively represents the amount of the loading fluid passing through each of the above-mentioned components. The individual droplet-forming unit 209 further comprises a bypass channel 208 located around the droplet-forming well 205.

The inflow channel 203 of the droplet-forming unit 209 is configured to accept the loading fluid from the inlet 201, and is in fluid communication with the neck channel 204, as exemplified in FIG. 2. In certain embodiments, the inflow channel 203 is also in fluid communication with the bypass channel 208. In certain embodiments, the loading fluid passing through the inflow channel 203 is divided into two streams: one of the two streams, i.e. a first portion of the loading fluid, enters the neck channel 204, and the other stream, i.e. a second portion of the loading fluid, enters the bypass channel 208. The neck channel 204 and the bypass channel 208 are configured to have a cross-sectional width ratio of the bypass channel 204 to the neck channel 208, the cross-sectional width ratio being selected so that the first portion of the loading fluid fills the droplet-forming well 205 before the second portion of the loading fluid fills the bypass channel 208. As such, the space of the droplet-forming well 205 can be fully utilized and the size of a droplet formed in a droplet-forming well 205 can match the theoretical volume of a droplet-forming well 205 to ensure maximized sample input size. In certain embodiments, the cross-sectional width ratio of the bypass channel to the neck channel is approximately 0.2 to approximately 1.0, approximately 0.3 to approximately 0.9, approximately 0.4 to approximately 0.8, approximately 0.5 to approximately 0.7, or approximately 0.6. In certain embodiments, the cross-sectional width ratio of the bypass channel to the neck channel is approximately 0.45 to approximately 0.85, approximately 0:55 to approximately 0.75, or approximately 0.65. In certain embodiments, the cross-sectional width ratio of the bypass channel to the neck channel is approximately 0.75. Other cross-sectional width ratios of the bypass channel to the neck channel that allows the first portion of the loading fluid to fill the droplet-forming well before the second portion of the loading fluid fills the bypass channel are also within the contemplation of the present disclosure.

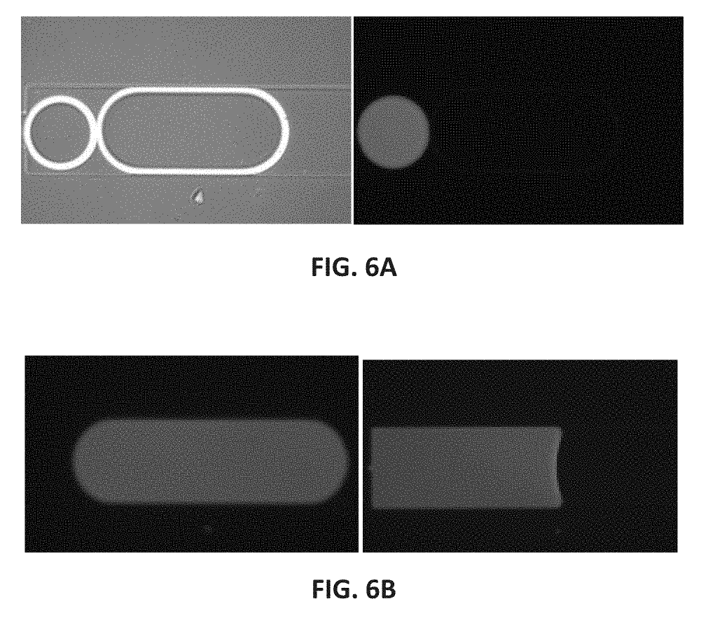

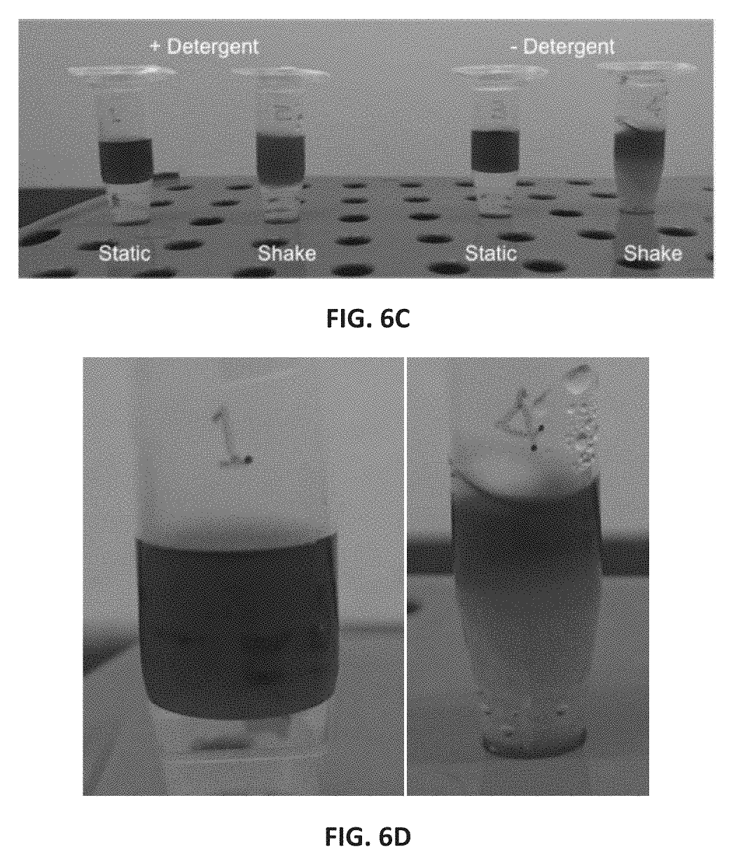

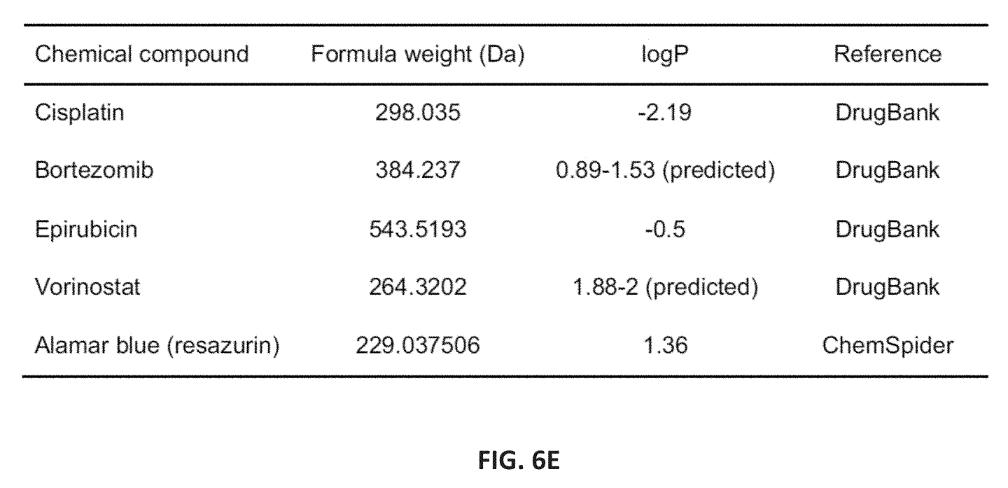

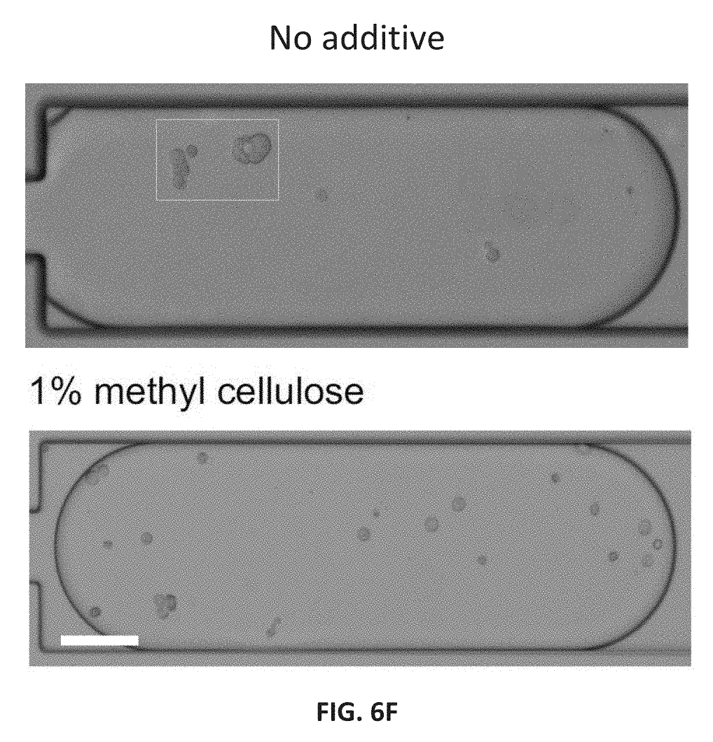

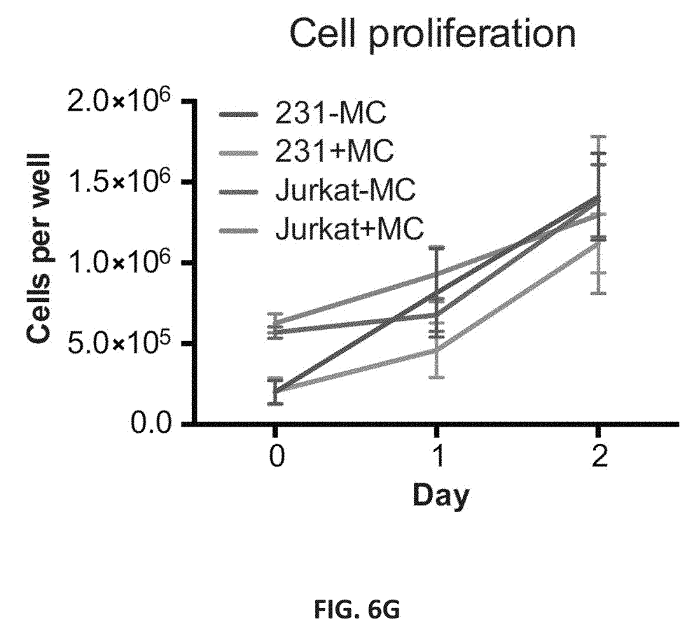

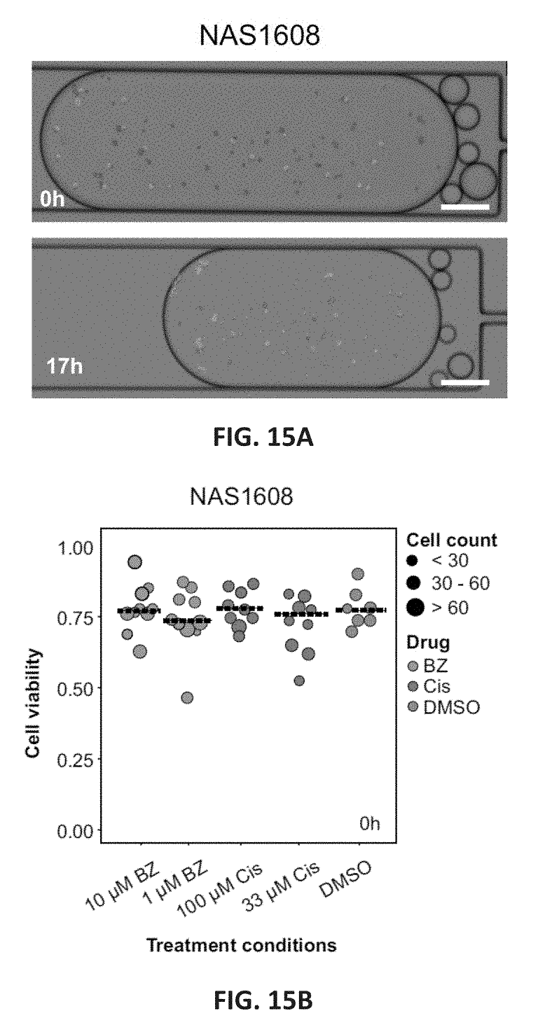

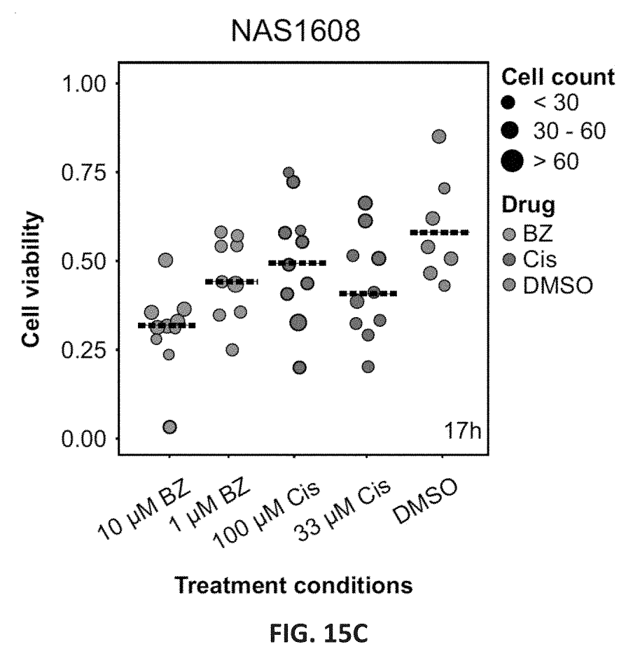

The loading fluid comprises a sample fluid and a carrier fluid in distinct layers separated by an interface. In certain embodiments, the loading fluid has a carrier fluid, a sample fluid A, the carrier fluid, and a sample fluid B, all of which are sequentially arranged in a loading chamber along the direction of ejecting the loading fluid. When the loading fluid is ejected from the loading chamber and is infused into the droplet-forming channel 200 through the inlet 201, the sample fluid B will first reach the inlet 201, followed by the carrier fluid, followed by the sample fluid A, and then followed by the carrier fluid. The loading chamber can hold two or more sample fluids, which are segregated by a carrier fluid between two adjacent sample fluids. As such, the droplet formed in a droplet-forming well 205 comprises a shell formed by the carrier fluid encompassing a sample fluid therewithin, as exemplified in FIGS. 6F and 15A.

The carrier fluid can be a mixture comprising an oil and a surfactant, which facilitates the formation of droplets in the droplet-forming wells 205, segregates two distinct sample fluids in the loading chamber, prevents the coalescence of two droplets of two distinct sample fluids, and helps maintain constant drug concentrations within the droplets.

The neck channel 204 is in fluid communication with the droplet-forming well 205 for delivering a first portion of the loading fluid from the inflow channel 203 to the droplet-forming well 205, as exemplified in FIG. 2. In certain embodiments, the neck channel 204 is configured to have a cross-sectional width that is smaller than the cross-sectional width of the droplet-forming well 205, so as to create an energy barrier to prevent a droplet from escaping from the droplet-forming well 205. In certain embodiments, the neck channel 204 has a cross-sectional width of approximately 50 μm to approximately 150 μm, approximately 60 μm to approximately 140 μm, approximately 70 μm to approximately 130 μm, approximately 80 μm to approximately 120 μm, or approximately 90 μm to approximately 110 μm. In certain embodiments, the neck channel 204 has a cross-sectional width of approximately 65 μm to approximately 155 μm, approximately 75 μm to approximately 145 μm, approximately 85 μm to approximately 135 μm, approximately 95 μm to approximately 125 μm, approximately 105 μm to approximately 115 μm. In certain embodiments, the neck channel 204 has a cross-sectional width of approximately 75 μm. In certain embodiments, the droplet-forming well 205 has a cross-sectional width of approximately 100 μm to approximately 500 μm, approximately 150 μm to approximately 450 μm, approximately 200 μm to approximately 400 μm, or approximately 250 μm to approximately 350 μm. Other cross-sectional widths of the droplet-forming well 205 are also within the contemplation of the present disclosure. In certain embodiments, the droplet-forming well 205 has a cross-sectional width of approximately 300 μm. In certain embodiments, the neck channel 204 has a cross-sectional width of approximately 100 μm, and the droplet-forming well 205 has a cross-sectional width of approximately 300 μm. Other cross-sectional widths of the neck channel 204 and the droplet-forming wells 205 are also within the contemplation of the present disclosure.

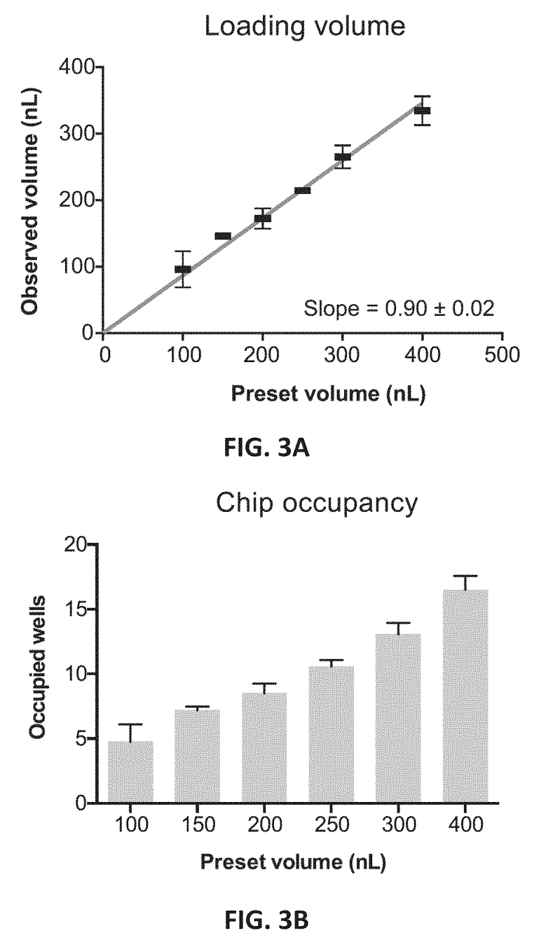

The incorporation of the neck channel 204 into a droplet-generating microfluidic chip 100 and the configuration of the neck channel 204 with respect to the droplet-forming well 205 can significantly improve the robustness and/or stability of a droplet formed within the droplet-forming well 205, restrict crosstalk with subsequent flow during sample loading and prevent droplet escape during overnight incubation at 37° C. In particular, FIG. 3A shows that there was good correlation between the observed loaded volume and the preset loading volume. The observed loaded volume was calculated by multiplying the number of droplet-forming wells 205 that are occupied by a sample fluid (at 0.5 increments) by the theoretical well volume (length×width×height) of each droplet-forming well 205 according to certain embodiments of the present disclosure, and the preset loading volume referred to the volume of a sample fluid set on a loading chamber. The good linearity shown in FIG. 3A indicates that almost all of the sample fluid can be loaded into the droplet-forming wells 205 and there was minimal loss of the sample fluid, which means that droplets formed in the droplet-forming units 209 are robust.

Furthermore, the fidelity of the observed loaded volume as compared to the preset loading volume allows the adjustability of the number of screening conditions, since the total number of screening conditions can be calculated by dividing the total number of droplet-forming wells 205 on a droplet-forming channel 200 by the number of the occupied wells, which can be predicted based on the preset loading volume of a sample fluid, as shown in FIG. 3B. For example, if 200 nL of the sample fluid is loaded onto a droplet-forming channel having 48 droplet-forming wells, 8 droplet-forming wells will be occupied based on the bar graph shown in FIG. 3B, and this allows 6 screening conditions to be tested; if 100 nL of the sample fluid is loaded onto a droplet-forming channel having 48 droplet-forming wells, 5 droplet-forming wells will be occupied based on the bar graph shown in FIG. 3B, and this allows 9-10 screening conditions to be tested. As such, the number of screening conditions can be flexibly adjusted by adjusting the volume of the sample fluid in a loading fluid thanks to the fidelity of the loaded sample fluid conferred by the neck channel 204. Such flexibility in adjusting the number of screening conditions can hardly be achieved by existing droplet-generating microfluidic chips due to the loss of sample fluids during droplet formation.

The restricted flow port element 206 is in fluid communication with both the droplet-forming well 205 and the bypass channel 208, and is configured to generate a restricted flow to facilitate droplet formation in the droplet-forming well 205, as exemplified in FIG. 2. By the term “restricted flow” in this context, it is meant that the restricted flow port element 206 offers restriction to a flow therethrough, into the bypass channel 208 from the droplet-forming well 205, in association, for example, with a higher pressure volume. This restriction in the restricted flow port element 206 can be provided, for example, by having filter media positioned over or in the restricted flow port element 206, and/or by limiting the cross-sectional dimension of the restricted flow port element 206. In certain embodiments, the restricted flow port element 206 is a restriction channel having a cross-sectional with of approximately 5 μm to approximately 20 μM, approximately 6 μm to approximately 19 μm, approximately 7 μm to approximately 18 μm, approximately 8 μm to approximately 17 μm, approximately 9 μm to approximately 16 μm, approximately 10 μm to approximately 15 μm, approximately 11 μm to approximately 14 μm, or approximately 12 μm to approximately 13 μm. In certain embodiments, the restricted flow port element 206 is a restriction channel having a cross-sectional width of approximately 15 μm. Other reasonable cross-sectional widths of the restriction channel are also within the contemplation of the present disclosure.

The bypass channel 208 is located around the droplet-forming well 205, and is configured to deliver a second portion of the loading fluid from the inflow channel 203 to the outflow channel. In certain embodiments, the bypass channel 208 of a droplet-forming unit 209 starts from the intersection between the inflow channel 203 and the neck channel 204, and ends at the intersection between the restricted flow port element 206 and the outflow channel 207, as illustrated in FIG. 2.

### Fabrication of the Microfluidic Chip

In certain embodiments, the microfluidic chip 100 can be prepared by a) providing a mold comprising complementary features to the microfluidic chip 100 with features described above; b) contacting the mold with a polymer liquid and then solidifying the polymer liquid to form a mold covered by a layer of the solidified polymer; c) detaching the solidified polymer from the mold to obtain the microfluidic chip 100. The mold can be made from a material selected from a group consisting of crystalline silicon, amorphous silicon, glass, quartz, and metals. Other materials having similar properties are also within the contemplation of the present disclosure. In certain embodiments, the mold is a silicon wafer having complementary features to the microfluidic chip 100 as described above. The polymer used to prepare the microfluidic chip 100 includes, but not limited to, poly(dimethylsiloxane) (PDMS), poly(methyl methacrylate), polyethylene, polyetheretherketone, polyurethane, polypropylene, polyimide, polystyrene, hydrogel, polycarbonate, and combinations thereof. Other polymers having similar properties are also within the contemplation of the present disclosure. In certain embodiments, the polymer used to fabricate the microfluidic chip 100 is PDMS, because of its ease of fabrication, transparency and biocompatibility.

Microfluidic Chip Design and Fabrication and Assembly of a Microfluidic System

Soft photolithography by photomask (Shenzhen Newway, China) was used to fabricate SU-8 negative photoepoxy (Microchem, USA) on silicon wafer (Harbin Tebo Technology, China) following standard procedures to make the patterned wafers. The patterned wafers used in this study were determined to be 62-78 μm in height using KLA-Tencor AlphaStep D-600 Stylus Profiler (KLA-Tencor, USA).

Polydimethylsiloxane (Dow Corning, USA) at 1:7 ratio (w/w) base to curing agent ratio (w/w) was poured onto the patterned wafers, baked in an oven at 65° C. for 25 min, and peeled off to generate PDMS slabs. Lastly, the PDMS slabs were plasma bound to 2.4×2.4 cm No. 1.5 square glass coverslips using Harrick Plasma PDG-002 Expanded Plasma Cleaner (Harrick Plasma, USA) to generate the ready-to-use microfluidic systems after baking at 65° C. overnight.

In certain embodiments, the restricted flow port element 206 of the microfluidic chip 100 has a transverse dimension of approximately 15 μm×150 μm, the droplet-forming well 205 of the microfluidic chip 100 has a transverse dimension of approximately 300 μm×1150 μm, and the neck channel 204 of the microfluidic chip 100 has a transverse dimension of approximately 100 μm×225 μm. Other suitable dimensions are also within the contemplation of the present disclosure.

### Using the Microfluidic System in a Drug Screening Assay or a Method for Drug Screening

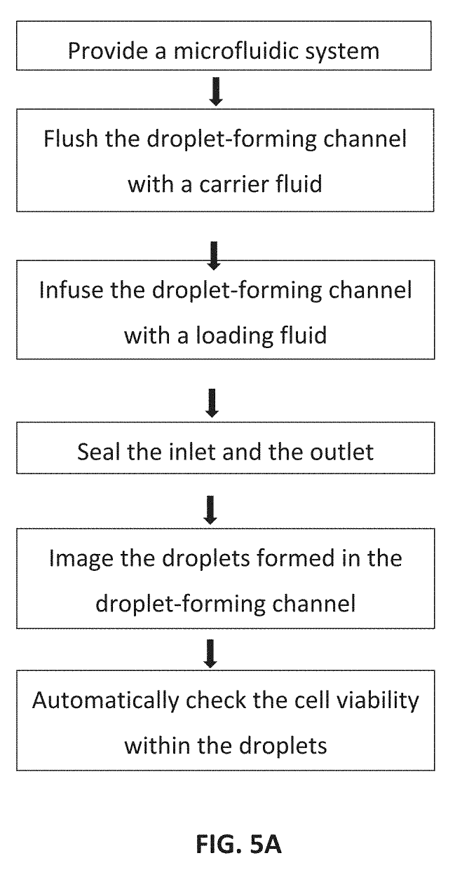

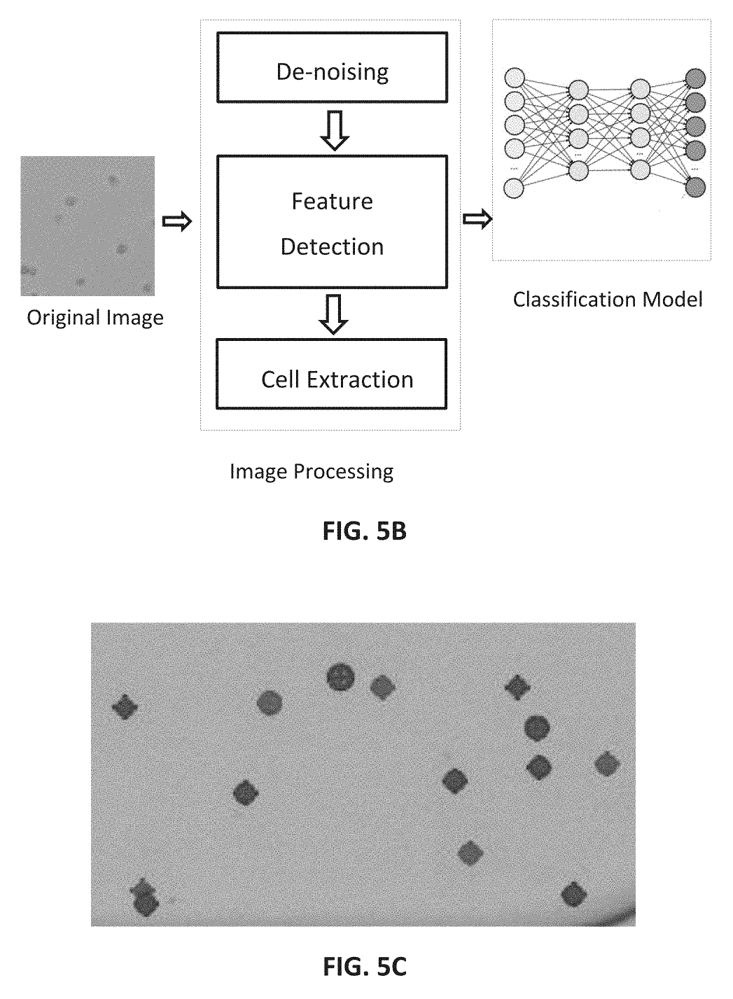

A complete drug screening assay or method for drug screening will now be described with reference to the microfluidic system of the present disclosure, as shown in FIG. 5A.

Preparation of Loading Fluid

In certain embodiments, the loading fluid is prepared by sequentially withdrawing a carrier fluid followed by a sample fluid, segregated by a carrier fluid before withdrawing another sample fluid into a loading chamber. In certain embodiments, the loading chamber can be a tubing. In other embodiments, the loading chamber can be a tubing that is connected on one end with a pressure-asserting device, e.g. a syringe pump, and connected to the inlet of the microfluidic system on the other end. Any other loading chambers that are compatible with the microfluidic system are also within the contemplation of the present disclosure.

In certain embodiments, the sample fluid can be a mixture comprising cells, a drug, a cell culture medium, an additive, a dead cell indicator, and/or a metabolic indicator. As such, drug concentration can be freely adjusted during premixing with cells before loading on chip. In certain embodiments, the cells are cancer cells. In certain embodiments, the cancer cells can be cancer cell lines, primary tumor cells, secondary tumor cells, cancer stern cells, or circulating tumor cells. In certain embodiments, the cancer cell line can be Jurkat E6.1 cells, MDA-MB-231 cells, or NAS1604C that is derived and amplified from the primary tumor cell NAS1604 in the present disclosure. In certain embodiments, the primary tumor cells can be obtained from primary tumors from human patients. In certain embodiments, the primary tumor is nasopharyngeal tumor, colon carcinoma, prostate cancer, breast cancer, lung cancer, skin cancer, liver cancer, bone cancer, ovary cancer, pancreas cancer, brain cancer, head cancer, neck cancer, lymphoma, leukemia, or brain cancer. Any other solid tumors are also within the contemplation of the present disclosure. The secondary tumor cells are cells obtained from secondary tumors, and secondary tumors are cancers that have spread and/or metastasized from the place where it first started to another part of the body. For example, cancer cells may spread from the breast (primary cancer) to form new tumors in the lung (secondary tumor). Cancer stem cells are cancer cells found within tumors or hematological cancers that possess characteristics associated with normal stem cells, specifically ability to give rise to all cell types found in a particular cancer sample.

In certain embodiments, the suspended cancer cell line is Jurkat E6.1 cells, derived from human acute T cell leukemia. Other suspended cancer cell lines are also within the contemplation of the present disclosure. In certain embodiments, the adherent cancer cell line is MDA-MB-231 cells, derived from human metastatic breast adenocarcinoma. Other adherent cancer cell lines are also within the contemplation of the present disclosure. In certain embodiments, the primary tumor cells can be dissociated from primary nasopharyngeal tumors from human patients. Other primary tumor cells are also within the contemplation of the present disclosure.

The cell culture medium can be any conventional culture medium suitable for a particular cell line. In certain embodiments, the cell culture medium contains v/v 1%-20% Fetal Bovine Serum (FBS). In certain embodiments, the volume to volume (v/v) percentage of FBS is 2%-19%, 3%-18%, 4%-17%, 5%-16%, 6%-15%, 7%-14%, 8%-13%, 9%-12%, or 10-11%. In certain embodiments, the volume to volume percentage of FBS is approximately 5%. FBS possesses emulsification properties that affect droplet formation and stability, and approximately 5% (v/v) FBS resulted in optimal droplet formation and stability.

In certain embodiments, the additive in the sample fluid prevents the clustering of adherent cell lines and improves the reliability of the automatic cell viability investigation. In certain embodiments, the additive is methyl cellulose, Pluronic® F-68, and Matrigel®. In certain embodiments, the additive is 0.5%-3% (m/v) methyl cellulose. In certain embodiments, the additive is 0.6%-2.5% (m/v), 0.7%-2.0% (m/v), 0.8%-1.9% (m/v), 0.9%-1.8% (m/v), 1.0%-1.7% (m/v) (m/v), 1.1%-1.6% (m/v), 1.2%-1.5% (m/v), or 1.3%-1.4% (m/v) methyl cellulose. In certain embodiments, the additive is methyl cellulose of approximately 1.0% (m/v). Any other additives and any other mass to volume percentages of the additives that prevent the clustering of adherent cell lines while do not affect droplet formation are also within the contemplation of the present disclosure.

In certain embodiments, the dead cell indicator can be ethidium homodimer 1, Alamar Blue, SYTOX Green nucleic acid stain, or propidium iodide. In certain embodiments, the metabolic indicator can be Calcein AM, C₁₂-resazurin, SYTO 10 dye, or SYBR 14 nucleic acid stain. In certain embodiments, the dead cell indicator is 2 μM ethidium homodimer 1. Any other dead cell indicators and/or metabolic indicators that do not affect cell viability at the time frame of drug screening are also within the contemplation of the present disclosure.

In certain embodiments, the carrier fluid can be a mixture comprising an oil and a surfactant or a detergent. The carrier fluid facilitates the formation of droplets in the droplet-forming wells 205, segregates two distinct sample fluids in the loading chamber, prevents the coalescence of two droplets of two distinct sample fluids, and helps maintain constant drug concentrations within the droplets. The oil can be any oil that is substantially inert under the screening conditions and substantially immiscible with water. The selection of the appropriate oil is well within the skill of a person of ordinary skill in the art. In certain embodiments, the oil is a perfluorinated alkane, a perfluorinated trialkyl amine, and/or a mixture thereof. In certain embodiments, the oil is a C₆-C₁₂, C₆-C₁₀, or C₈-C₁₂ perfluorinated alkane. In certain embodiments, the oil is C₆F₁₄, C₇F₁₆, C₈F₁₈, C₉F₂₀, C₁₀F₂₂, C₁₁F₂₄, C₁₂F₂₆ or a mixture thereof. In certain embodiments, the oil is C₁₀F₂₂, i.e. Fluorinert® FC-3283 oil. In certain embodiments, the oil is C₆F₁₀, i.e. Fluorinert® FC-72 oil. In certain embodiments, the oil is a perfluorinated trialkyl amine. In certain embodiments, the oil is (CₘFₘ₊₂)(CₙFₙ₊₂)(CₚFₚ₊₂)N, wherein each of m, n, and p is independently a whole number selected from 1-12. In certain embodiments, each of m, n, and p is independently a whole number selected from 1-6. In certain embodiments, the oil is perfluorinated tripentyl amine, i.e. Fluorinert® FC-70 oil. In certain embodiments, the oil is bisnonafluorobutyl trifluoromethyl amine, i.e. Fluorinert® FC-40 oil.

In certain embodiments, the carrier fluid is a mixture comprising an oil supplemented with approximately 1-5% (w/v) fluorosurfactant. In certain embodiment, the carrier fluid is a mixture comprising an oil supplemented with approximately 2% (w/v) fluorosurfactant. A fluorosurfactant is a synthetic organofluorine chemical compounds that have multiple fluorine atoms, which can be polyfluorinated or fluorocarbon-based perfluorinated). A fluorosurfactant has a fluorinated “tail” and a hydrophilic “head”, such as perfluorooctanesulfonic acid and pertluorooctanoic acid. In certain embodiments, the carrier fluid is a mixture comprising Fluorinert® FC-40 oil supplemented with approximately 2% 008-Fluorosurfactant (Ran Biotechnologies, USA). The selection of the appropriate fluorosurfactant is well within the skill of a person of ordinary skill in the art. The surfactant or detergent is added to prevent the cross-contamination between two adjacent droplets containing two different samples (FIG. 6A), to prevent the absorption of samples by the PDMS-based channel wall (FIG. 6B), and to enhance phase separation between the sample fluid, i.e. aqueous phase, and the carrier fluid, i.e. oil phase (FIGS. 6C and 6D). Any other surfactant or detergents at any other concentrations that exhibit the above-mentioned technical effects are also within the contemplation of the present disclosure.

### Assembling the Microfluidic System

In certain embodiments, the microfluidic system is assembled by attaching the microfluidic chip with a transparent substrate that is suitable for microscopic observation. The attachment between the microfluidic chip and the transparent substrate can be achieved by any existing methods, such as plasma treatment of the substrate. In certain embodiments, the transparent substrate can be a glass slide, a glass coverslip, or a glass-bottom dish. In certain embodiments, the transparent substrate is a glass coverslip. Any other transparent substrates, e.g. plastic substrates, that are suitable for cell culture, droplet formation, and can be used for microscopic observation are also within the contemplation of the present disclosure.

In certain embodiments, the inlet and outlet of the microfluidic system are connected with tubings that are further connected with a loading chamber. Any other intermediate elements that connect the microfluidic chip and the loading chamber are also within the contemplation of the present disclosure.

Flushing the Droplet-Forming Channel with a Carrier Fluid

In certain embodiments, the microfluidic system is flushed with the carrier fluid to remove any impurities within the newly assembled microfluidic system and to form a layer of oil phase at the wall of the droplet-forming channels. In certain embodiments, the microfluidic system is flushed with the oil phase from the outlet to the inlet. In certain embodiments, the microfluidic system is flushed with the oil phase at 500 μL/h by syringe pump.

Infusing the Droplet-Forming Channel with a Loading Fluid

After the microfluidic system is flushed with the oil phase, the loading fluid is infused to the microfluidic system to form droplets of the samples that have been pre-loaded in the loading chamber. In certain embodiments, the loading fluid is infused from the inlet, in certain embodiments, the infusing speed of the loading fluid is 10-60 μL/h. In certain embodiments, the infusing speed of the loading fluid is 15-55 μL/h, 20-50 μL/h, 25-45 μL/h, 30-40 μL/h, or 35 μL/h. In certain embodiments, the infusing speed of the loading fluid is 25 μL/h. Any other infusing speeds that do not affect the droplet formation and stability are also within the contemplation of the present disclosure.

In certain embodiments, the infusing of a loading fluid can be achieved by an autosampler or a syringe pump.

The inlet and outlet are sealed after the loading fluid is infused into the microfluidic chip, which is then placed in a humidified environment for further incubation.

Imaging and Automatic investigation of Cell Viability

In certain embodiments, the microfluidic system containing droplets of a plurality of samples treated with different drugs at different concentrations is placed under a microscope system for acquisition of images of the each sample under each condition within the droplets. In certain embodiments, one sample under one condition is enclosed in a serial array of consecutive droplets.

In certain embodiments, cell viability is carried out by counting the number of live cells and dead cells within a droplet either by labeling dead cells using a dead cell indicator, e.g. ethidium homodimer 1, Alamar Blue, SYTOX Green nucleic acid stain, or propidium iodide, or by labeling live cells using a metabolic indicator, e.g. Calcein AM, C₁₂-resazurin, SYTO 10 dye, or SYBR 14 nucleic acid stain. In certain embodiments, the investigation of cell viability can be manual or automated as shown in FIGS. 5B and 5C.

In certain embodiments, the microfluidic system can be further coupled with RNA sequencing library preparation for single cells for single-cell next generation sequencing (NGS). In certain embodiments, the microfluidic system can be further coupled with fluorescence activated cell sorting (FACS). Other applications that could help elucidate the molecular background of the investigated cancer to be coupled with the microfluidic system are also within the contemplation of the present disclosure.

Below are examples of using the microfluidic system assembled using the droplet-forming microfluidic chip 100 of the present disclosure in a drug screening assay. In certain embodiments, the microfluidic system can be used for monitoring responses of cancer cells or primary cells at different drug concentrations, or to efficiently and accurately determine drug efficacy based on a small sample input size. It should be understood, however, that the description is only for illustrative not limiting purpose. The microfluidic system of the present disclosure can be used for many other purposes.

Cancer Cell Lines and Cell Culture

Jurkat E6.1 cells (ATCC® TIB-152™) and MDA-MB-231 cells (ATCC® HTB-26™) were used as models for suspended and adherent cancer cell lines respectively. Jurkat cell line was derived from human acute T cell leukemia, whereas MDA-MB-231 cell line was derived from human metastatic breast adenocarcinoma.

Jurkat cells were cultured in Advanced RPMI 1640 medium (Life Technologies, USA) supplemented with 5% fetal bovine serum (FBS) (Gemini, USA), 100 U/mL Penicillin-Streptomycin (Life Technologies, USA), 2 mM L-glutamine (Life Technologies, USA), and 10 mM HEPES pH7.4 (Life Technologies, USA).

MDA-MB-231 cells were cultured in Dulbecco's Modified Eagle Medium (Life Technologies, USA) supplemented with 5% FBS, 100 U/mL Penicillin-Streptomycin and 2 mM L-glutamine.

All cells were cultured in humidified incubator at 37° C. supplemented with 5% CO₂.

Primary Tumor and Tumor Dissociation

All human studies were conducted with the approval of the Panel on Research Ethics of University of Macau and the Research Ethics Committee of Kiang Wu Hospital, according to the Materials Transfer Agreement between University of Macau and Kiang Wu Hospital. Informed consent for sampling and publication without identifiable information was obtained from all participating patients. All patient sample names were double encoded by the university and the hospital, respectively, to remove any trace of patient identity during sample collection, transfer, processing and analysis. Primary tumors were obtained from surgery conducted at Kiang Wu Hospital immediately after tumor resection. Tumor tissue was dissociated as previously described. Briefly, tumor tissue was first cut into small pieces by a scalpel, then transferred to a 50 mL conical tube containing 5 mL Digestion Buffer I (DMEM/F12 medium containing 5% FBS, 5 μg/mL insulin, 500 ng/mL hydrocortisone, 10 ng/mL epidermal growth factor (EGF), 20 ng/mL cholera toxin, 300 U/mL collagenase III and 100 U/mL hyaluronidase), and digested for no more than 12 h with shaking at 100 rpm in humidified incubator at 37° C. supplemented with 5% CO₂. After spinning down at 400 g at ambient temperature for 2 min, the cells were resuspended with 2 mL Digestion Buffer II (DMEM/F12 medium containing 5 mg/mL dispase II and 0.1 mg/mL deoxyribonuclease I), followed by digestion at ambient temperature for 5 min. The cells were then washed with 10 mL HBSS (Life Technologies, USA). 2 mL RBC lysis buffer (eBioscience, USA) was used to lyse red blood cells at ambient temperature for 3 min; this step was repeated until the solution becomes translucent. 12 mL HBSS (Life Technologies, USA) was finally added to stop the lysis. Dissociated cells were extracted by centrifugation of the filtrate through a 40 μm strainer (Falcon, USA). Lastly, the cells were resuspended in StemMACS iPS-Brew XF medium (Miltenyl Biotec, USA) and used for drug screening on chip.

On Chip Drug Screening Assay

All drugs used in this study were listed in Table 1, bortezomib and vorinostat were chosen as target drugs for leukemia, i.e. Jurkat cells, whereas cisplatin and epirubicin were chosen as target drugs for breast cancer, i.e. MDA-MB-231 cells. Another consideration of the chosen drugs was diverse therapeutic targets.

TABLE 1
Current microfluidic technologies for drug screening

| Sample | | Total sampled | |
|---|---|---|---|
| type | Subject | conditions | Technology |
| Protein | Enzyme | 704 | Droplet microfluidics |
| Cell | Cells in | 23 × 23 = 529 | Array printing |
| line | agarose | | |
| | Cells | 3328 | Nano-well patterning |
| | Bacteria | 20 | Flow microfluidics |
| | Suspended and | 2 channels of 5 | Droplet microfluidics |
| | adherent cells | conditions | of the present |
| | | | disclosure |
| Primary | Leukemia cells | 1266 | Plate reader assay^{#} |
| tumor | Lung cancer | 3 | Flow microfluidics |
| | and stromal | | |
| | cells | | |
| | Multiple | 1 drug × <5 dose | Flow microfluidics |
| | myeloma cells | | |
| | T2 breast | 16 | Implanted chip |
| | tumor | | |
| | Primary tumor | 5-10 conditions | Droplet microfluidics |
| | dissociated | depending on cell | of the present |
| | cells | number | disclosure |
| Cultured | Cultured CTC | 38 × 6 = 228 | Ex vivo culture |
| tumor/ | Cultured | Dependent on cell | Trap and release → in |
| CTC | single CTC | amplification | vitro culture |

^{#}This is not microfluidic-based assay.

On chip drug screening was performed using the PDMS-based microfluidic chip as described above. A 500 μL glass syringe (Hamilton, USA) and polytetrafluoroethylene (PTFE) tubings with appropriate bore (Cole Parmer, USA) was used to connect between the syringe pump (Harvard Apparatus PHD Ultra Syringe Pump, USA) and the microfluidic chip. Fluorinert® FC-40 (Sigma-Aldrich, USA) supplemented with 2% 008-Fluorosurfactant (Ran Biotechnologies, USA) was used as oil phase; relevant cell culture medium, supplemented with 1% (w/v) methyl cellulose (Sigma-Aldrich, USA) was used as aqueous phase. Cells treated with 0.1% dimethyl sulfoxide (DMSO) were used as negative control.

Briefly, cells at final concentrations of 1-2×10⁶ cells per mL were aliquoted in 0.2 mL PCR tubes, then mixed with corresponding drugs and 2 μM ethidium homodimer 1 (Life Technologies, USA) by manual pipetting. Next, 100-200 nt, cell-drug mixtures were loaded into the tubing, consecutively segregated by oil phase at withdrawal rate of 200 μL/h by syringe pump. After loading all mixtures, the tubing was inserted into the microfluidic chip, which was back-flushed with oil phase at 500 μL/h by syringe pump. After that, the mixtures were infused at 25 μL/h by syringe pump. Finally, the inlet and outlet tubings were cut and sealed with Vaseline (Vaseline, USA). The chips were placed in 150 mm cell culture dish (Coming, USA) containing wet paper towels, and transferred to humidified incubator at 37° C. supplemented with 5% CO₂ for 16-24 h incubation. Brightfield and red fluorescence images (Ex. 531/40 nm, Em. 593/40 nm) were taken under 10× magnification (Life Technologies EVOS FL Imaging System, USA).

Microtiter Plate Drug Screening Assay

Microtiter plate drug screening assays were carried out on 96-well clear round flat-bottom plates (Corning, USA) or 384-well white square flat-bottom plates (Corning, USA).

First, 5.0×10⁵ or 1.0×10⁵ cells were seeded per well for 96-well and 384-well plates respectively. Drugs were diluted with Dulbecco's phosphate-buffered saline (DPBS) (Life Technologies, USA), and subsequently added to achieve final drug concentrations as indicated on the graphs. Afterwards, the plates were transferred to humidified incubator at 37° C. supplemented with 5% CO₂ for 16-24 h incubation. Finally, Alamar Blue assay was used to measure cell viability. Fluorescence intensity (Ex. 560 nm, Em. 590 nm) using auto-cutoff was measured from bottom on plate reader (Molecular Devices SpectraMax MS Plate Reader, USA). Cells treated with 0.1% dimethyl sulfoxide (DMSO) were used as negative control, while no cells were added to blank control. All experiments were performed in triplicate for 96-well plates and in quadruplicates for 384-well relates.

Image Processing for on Chip Data Analysis

For on chip assays, brightfield and red fluorescence images were initially processed by ImageJ v.1.50i, Cell counting was either performed manually or by Matlab v.R201.5a based on the workflow shown on FIG. 5B. Briefly, cells from brightfield and red fluorescence images were detected separately using a heuristic Hough Transformation model based on threshold implementation on circular diameter and pixel intensity. Next, each recognized cell was dissected into 10×10 pixels matrix for analysis of its pixel intensity. Subsequently, two layers of multi-radii analysis of pixel intensity around the center of each matrix distinguished signal from noise. Afterwards, cells were distinguished from noise based on the brightfield image, whereas cell viability was determined by signal intensity of corresponding red fluorescence image. Finally, positional information (defined by row: x, and column: y) and size (defined by radius: r) of discrete image matrix was used to classify each cell as “live” or “dead”. The total number of cells in each image corresponding to each droplet formation well on chip was summarized as a table in CSV format for calculation of cell viability. To ease application, we have developed a graphical user interface (GUI) for funning on Matlab.

Cell Viability Calculation

For on chip assays, the number of cells was counted in brightfield and red fluorescence images from each well respectively. Cell viability was calculated as follows:

$$Cell viability = \frac{( Total cells - Dead cells )}{Total cells} \qquad ( 1 )$$

where Total cells and Dead cells referred to the total number of cells counted from brightfield and red fluorescence images, respectively.

For normalized cell viability, mean cell viability of all sample wells were normalized to mean cell viability of all negative control wells.

$$Normalized cell viability = \frac{Sample}{DMSO control} \qquad ( 2 )$$

where Sample and DMSO control represented mean cell viability of sample and negative control wells respectively.

For microliter plate assays, average relative fluorescence signal measured by plate reader from 6 reads of each well was used as raw data point. Cell viability was calculated as follows:

$$Cell viability = \frac{Sample - Blank}{DMSO control - Blank} \qquad ( 3 )$$

where Sample represented raw data points of each sample well, whereas DMSO control and Blank represented average raw data points of all DMSO control and Blank wells, respectively.

Bar graphs and line plots were drawn by GraphPad Prism 5.1. Scatter plots were drawn by R v.3.3.2 using custom scripts. Figures were prepared by assembling images, graphs and plots using Adobe® Illustrator® CS6 v.16.0.0.

### EXAMPLE 1

Improvement of Microfluidic Chip Design and Validation

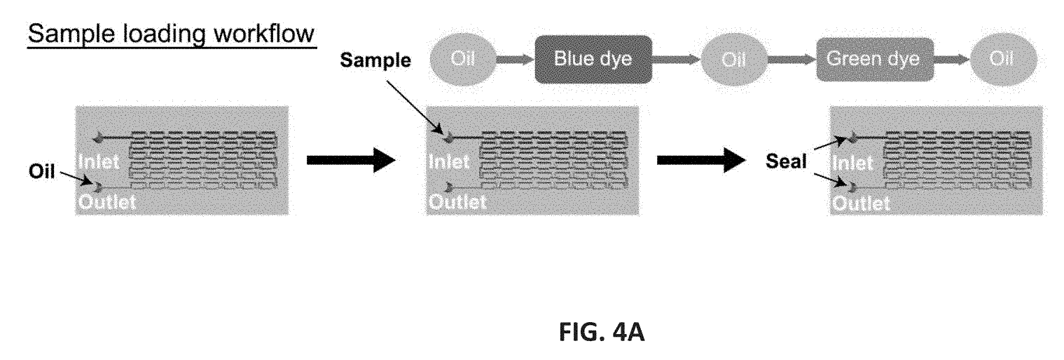

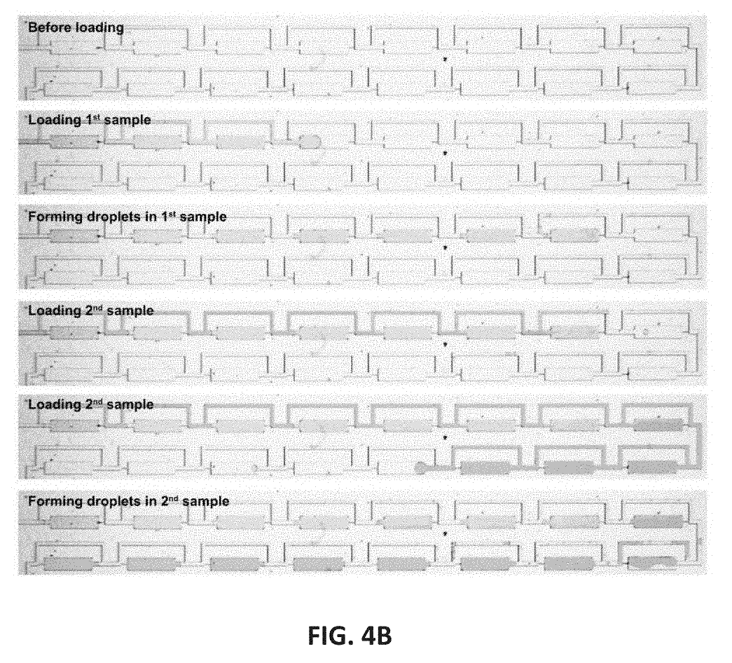

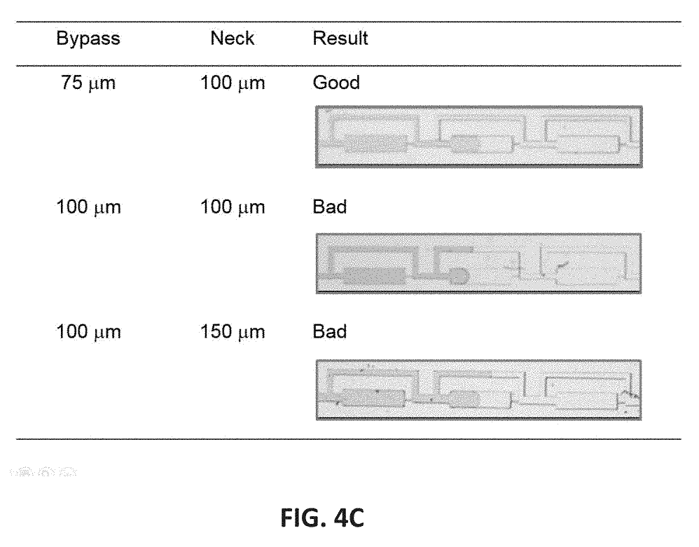

In this example, the microfluidic chip is fabricated from PDMS, and contains 2 droplet-forming channels 200, wherein each droplet-forming channel 200 has 6 rows and each row contains 8 droplet-forming units 209, wherein each droplet-forming unit 209 has a droplet-forming well 205, an inflow channel 203, neck channel 204, a restriction channel 206, an outflow channel 207, and a bypass channel 208, and two adjacent droplet-forming units 209 are connected by the outflow channel of the preceding unit and the inflow channel of the succeeding unit (FIG. 2). As such, each of the droplet-forming channel of the microfluidic chip in this Example contains 48 droplet-forming wells, and each microfluidic chip in this Example contains 96 droplet-forming wells (FIG. 1). Each droplet-forming channel 200 further has an inlet 201 and an outlet 202. The inlet 201 is connected with a loading chamber through an inlet tubing to receive a loading fluid and to provide the loading fluid to the droplet-forming units. All sample fluids within the loading fluid flowed in one direction and in sequential order as loaded in the inlet tubing (FIG. 4). Droplets were formed when aqueous solution filled the well and cut off from the bulk solution by subsequent incoming carrier fluid, e.g. oil.

Within the droplet-forming unit 209, the neck channel 204 is designed to work as a droplet back flow restriction (FIG. 2), by increasing the energy cost for droplet escape from the droplet-forming well 205. Droplets tend to escape from the droplet-forming wells 205 especially during overnight incubation at elevated temperature of 37° C. as compared to room temperature of 25° C. during sample loading. Therefore, the incorporation of the neck channel 204 into the droplet-forming channel, which allows the microfluidic chip to be used for overnight cell incubation, as such time frame is often required in order to reliably test a drug concentration on the viability of certain cells. In addition, the oil trapped in the neck channel 204 helped to prevent the stored droplet from coalescence with subsequent incoming droplets in the main channel. The physical separation of droplets between wells was designed to prevent cross-contamination between drug treatment conditions in different droplets. Furthermore, the droplet-forming wells 205 also acted as reference grids for image acquisition. On the other hand, the width ratio of the bypass channel 204 to the neck channel 208 was optimized to 3:4 in order to favor fluid flow into the droplet-forming well 205 over the bypass channel 208. In this work, three designs were tested: the widths of the bypass channel to the neck channel were 75 μm/100 μm, 100 μm/100 μm and 100 μm/150 μm, respectively. Results showed that the design of 75 μm/100 μm neck to bypass width favored fluid flow into the well over the bypass channel (FIG. 4C), which ensured full well filling in order to maximize channel space usage.

The robustness of the droplet formation was tested by checking the preservation of the loaded sample volume in the droplet-forming wells. The loading fluid was loaded with a syringe pump, and a good correlation between the observed volume or observed loaded volume and the preset volume or theoretical loading volume as preset on the syringe pump was observed (FIG. 3A). The observed volume was calculated by multiplying the number of droplet-forming wells 205 that are occupied by a sample fluid (at 0.5 increments) by the theoretical well volume (length×width×height) of each droplet-forming well 205, and the preset volume referred to the volume of a sample fluid set on the syringe pump.

The reliability of the droplet-forming microfluidic chip in droplet formation and separation was also tested using food dyes of different colors. In this experiment, the food dyes were dissolved in water, representing the sample fluid or aqueous phase; the carrier fluid or oil phase comprised Fluorinert® FC-40 oil supplemented with 2% 008-Fluorosurfactant. The sample loading workflow was illustrated in FIG. 4B. Firstly, the microfluidic chip was loaded with oil. Next, food dyes of blue and green colors were segregated by translucent oil phase and loaded consecutively onto the chip. Each dye formed droplets in sequential order that was identical to the loading sequence (FIG. 4A). As shown in FIG. 6A, addition of 2% 008-Fluorosurfactant (Ran Biotechnologies, USA) in Fluorinert® FC-40 oil (Sigma-Aldrich, USA) prevented droplets in contact from coalescence and cross-contamination, as no red fluorescence was Observed in the right droplet even when it touched the left droplet containing strong red fluorescence under illumination at 80% light intensity (Life Technologies EVOS FL Imaging System, USA), indicating that no cross-contamination between droplets occurred between droplets in contacts. Hence, these results demonstrated that testing multiple conditions in a single channel on chip is feasible.

Taken together, these results proved that the microfluidic chip is applicable for multi-drug conditions screening on a single channel with high flexibility based on sample input size and throughput requirements.

### EXAMPLE 2

Drug Screening Platform Setup and Optimization

The microfluidic chip validated in Example 1 was used for cell-based drug screening. The following criteria were considered: (1) the drug screening platform should maintain cell viability under investigated conditions; (2) the method is robust and well-controlled; (3) the readout is accurate and reproducible; and (4) the system is versatile for different cell culture systems.

Firstly, we optimized the carrier fluid or oil phase to assure cell viability on chip for a minimum of 7 days for cancer cell lines. Commercial oils including Fluorinert® series oils, e.g. fluorocarbon oils, silicone oil, and mineral oil in combination with different surfactants (either mixed with oil phase or aqueous phase) at various concentrations have been tested. Among these, Fluorinert® FC-40 supplemented with 2% 008-Fluorosurfactant by Ran Biotechnologies provided optimal properties in terms of viscosity, volatility and droplet stability. Fluorosurfactant was added to prevent droplets from coalescence and cross-contamination of droplet contents when they touched each other (FIG. 6A). In addition, fluorosurfactant-emulsified droplets prevented fluorescent dye from adsorbing to the hydrophobic PDMS walls of the channel (FIG. 6B) or migrating into the oil phase (FIGS. 6C and 6D), in order to maintain constant drug concentrations in droplets. As shown in FIG. 6B, fluorosurfactant-emulsified droplets exhibited no fluorescent dye adsorption on PDMS walls of the channel after 15 h incubation at 37° C. Droplets containing red fluorescent dye were incubated at 37° C. for 15 h, but no trace of fluorescent dye was observed along the PDMS walls as the droplet shrank due to evaporation. As shown in FIGS. 6C and 6D where a phase separation experiment using Alamar Blue dye in phosphate buffered saline (PBS) as aqueous phase and Fluorinert® FC-40 oil as oil phase with and without fluorosurfactant (detergent) addition was performed in a CO₂ incubator (Static) or in a bacterial shaker with 200 rpm shaking (Shake). Results showed that Tube 1 (Static+Detergent) that mostly mimicked droplet state on chip prevailed clean phase separation after overnight incubation, whereas shaking enhanced dye migration into the oil phase in Tube 2 (Shake+Detergent) and to ga greater extent in absence of fluorosurfactant in Tube 4 (Shake−Detergent). Comparison of the formula weight and log P values of the drugs used in this assay and those of Alamar blue dye suggested that the observation from this experiment was likely to be relevant to the drugs.

Although droplet shrinkage due to evaporation after overnight incubation might affect drug concentration, it was assumed that all droplets on the same chip had equal evaporation rate, so results should be comparable. This problem would be addressed in future design by continuous perfusion. On the other hand, the Droplet Generation Oil for Probes by Bio-Rad was also good for droplet generation, but its high volatility rendered it suboptimal for long-term cell culture on PDMS-based chips. Other oils and surfactant combinations were not used due to adverse properties: (1) the investigated oils in absence of surfactant provided poor droplet generation efficiency on our chip; (2) mineral oil had high viscosity that hindered its loading on chip; (3) surfactants like Triton X-100 and sodium dodecyl sulfate (SDS) compromised cell viability at all tested concentrations; and (4) the Pluronic® series surfactants did not alter droplet generation efficacy, nor enhanced adherent cells to remain in suspension.

For the aqueous phase or the sample fluid, applying optimal cell culture medium for cell survival is critical. Next comes the consideration of droplet generation. In this study, three culture medium recipes, namely Dulbecco's Modified Eagle Medium (DMEM), Advanced RPMI 1640 and StemMACS iPS-Brew XF medium, have been tested to be feasible for drug screening on chip. Other medium recipes should also be feasible because mammalian cells, in general, require similar ionic strength, which is a major consideration factor for chip performance. Fetal bovine serum (FBS) frequently used in mammalian cell culture possesses emulsification properties that affect droplet formation and stability. In this study, it was found that 5% FBS was optimal, but empirical testing is recommended due to potential variance between products. Alternatively, additives should be added with caution. In our trial experiments, cell culture additives like 1% methyl cellulose, 0.1% Plutonic® F-68, and 8mg/mL Matrigel® did not affect droplet formation (data not shown).

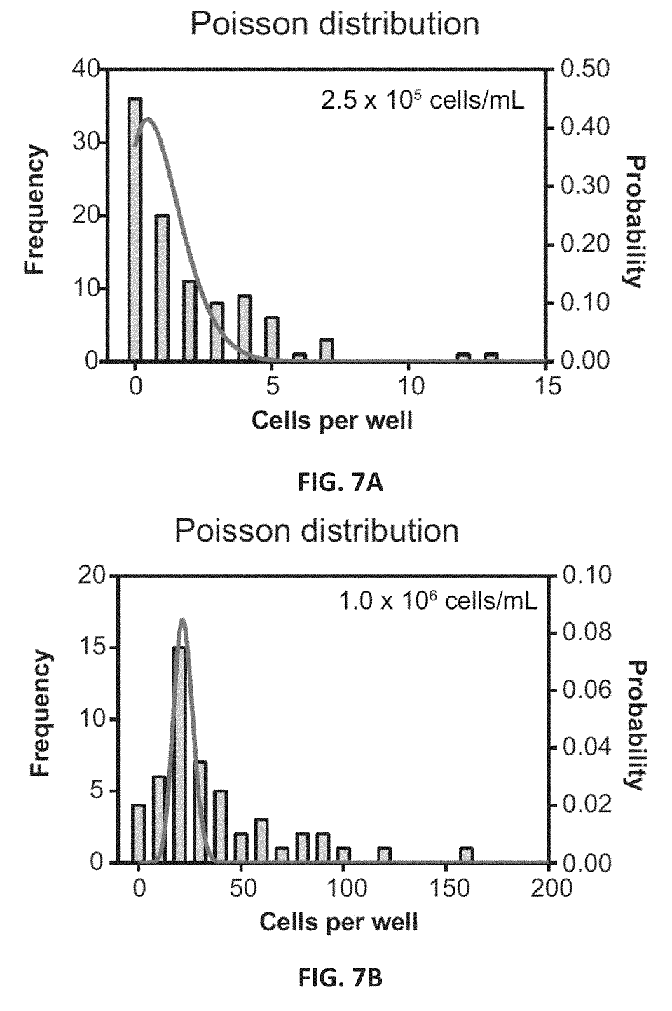

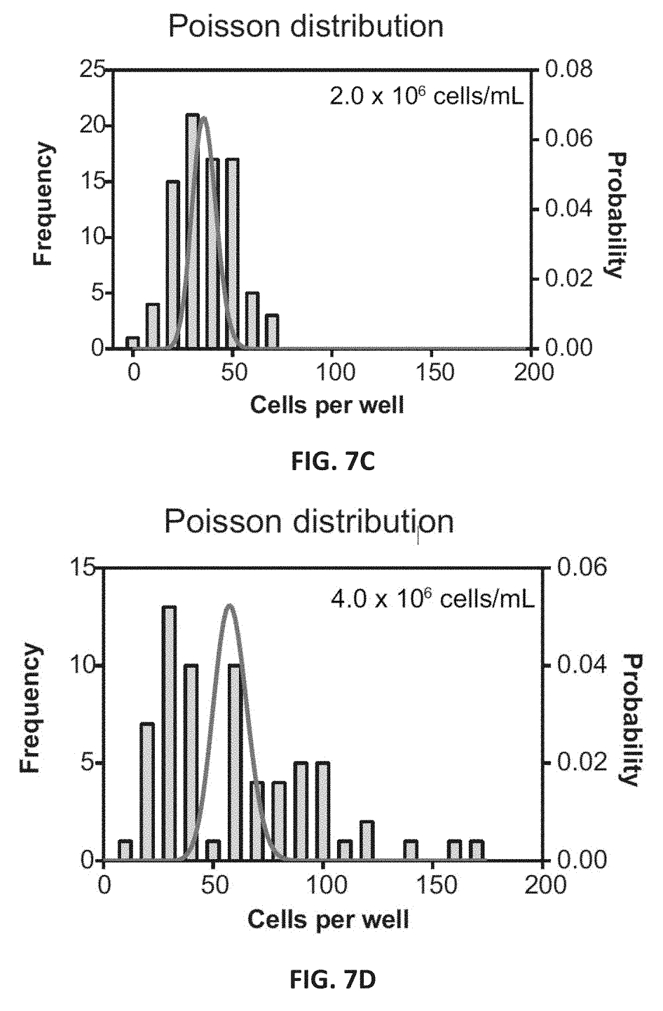

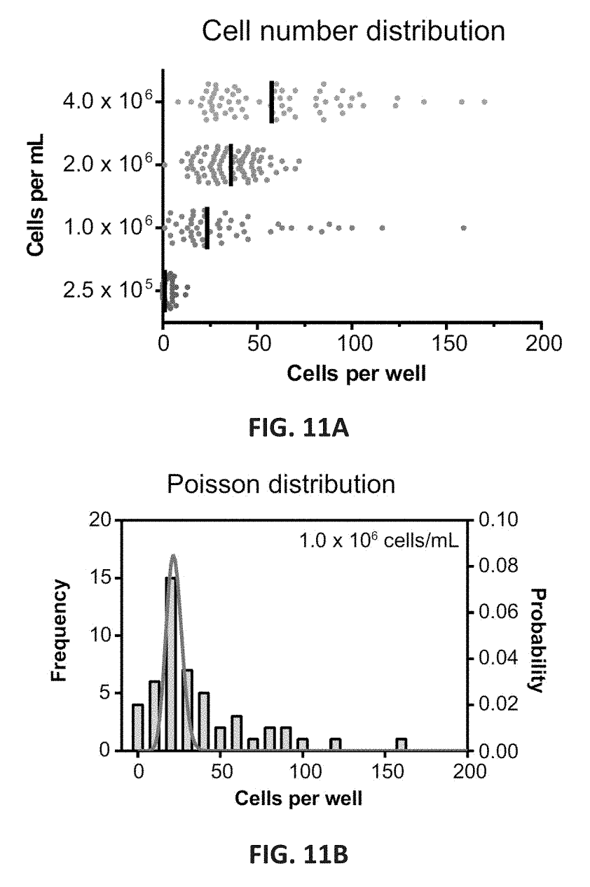

Secondly, optimal cell density on the chip was tested. Optimal cell density was considered based on two reasons: (1) sufficient cell population for statistical analysis of drug susceptibility, and (2) optimization of droplet cell density to avoid overcrowding. Overcrowded droplets led to cell aggregates, resulting in poor cell shredding during image processing and hence collapse of intelligent solution. By loading gradient concentrations of cells on chip, results manifested that there was positive correlation between the average number of cells per well and the concentration of cells before loading on chip (FIG. 11A), Nevertheless, as reported by other studies, Poisson distribution of cells per droplet prevailed in all wells regardless of the actual cell concentration used (FIGS. 7 and 11B). Consideration for cell concentration mainly involves cell size, which affects the volume occupied in each droplet. Consequently, 1×10⁶ cells/mL was used for MDA-MB-231 cells (average diameter 20 μm) while 2×10⁶ cells/mL was used for Jurkat cells (average diameter ˜10 μm). As for primary tumor cells, the cells were applied at a concentration range of 1-3×10⁶ cells/mL with a loading volume of 200 nL for each treatment condition. This ensured a sample cell population of over 100 cells for each treatment condition without compromising cell counting due to cell aggregation. Taken together, the assay enabled flexible adjustment of cell concentration and loading volume to achieve predictable sample cell population for each screening condition. Generally, 1-2×10⁶ cells/mid works for the majority of mammalian cells.

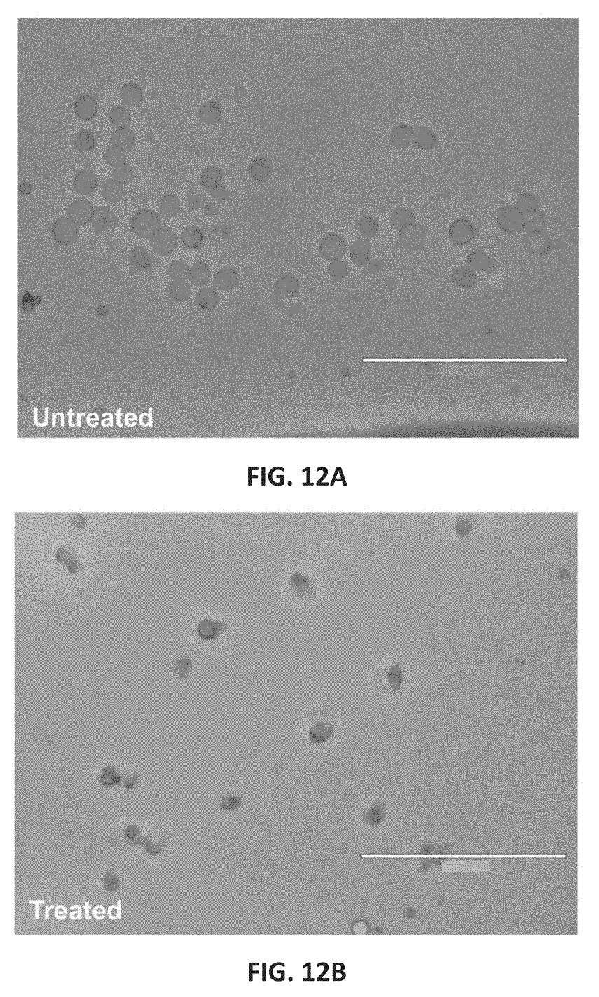

Thirdly, different cell viability indicator dyes were tested for staining efficiency, indicator reliability and cytotoxicity on chip. Since the sample fluid was premixed before loading to the chip, we tested for different cell viability indicator dyes and variant concentrations to ensure that the indicator dye had no impact on cell viability, at least within the time frame of the drug susceptibility test. Eventually, ethidium homodimer 1 staining was adopted to indicate dead cells. Ethidium homodimer 1 is a cell-impermeable, high affinity nucleic acid stain emitting strong red fluorescence after binding to DNA. It gave strong red fluorescence after cells were treated with drug but not DMSO) (FIG. 12). Time-lapse imaging of cells incubated with 2 μM ethidium homodimer 1 indicated that the dye did not affect cell death and proliferation within 48 h post-treatment (data not shown),

Lastly, we tried to make our drug screening platform versatile for different kinds of cancers. Because cells were suspended in droplets during drug treatment, adherent cells formed aggregates in the absence of physical support (FIG. 6F). Therefore, we tried to emulsify cells with detergents, maintain cells in semi-solid matrix using Matrigel®, increase fluid viscosity using sucrose, etc, but failed. Eventually, we found that culture medium supplemented with 1% methyl cellulose maintained cell viability on chip and did not affect cell proliferation off chip for both suspended and adherent cells (FIG. 6G). Methyl cellulose has been applied to grow 3D organoid cultures of human pluripotent stem cells (HPSCs) in vitro. Our observation of its success was reduced cell movement inside droplets, thus preventing cells from touching and maintained them in suspension. Hence, we used this condition for subsequent drug screening experiments.

### EXAMPLE 3

Drug Screening of Suspended and Adherent Cancer Cell Lines

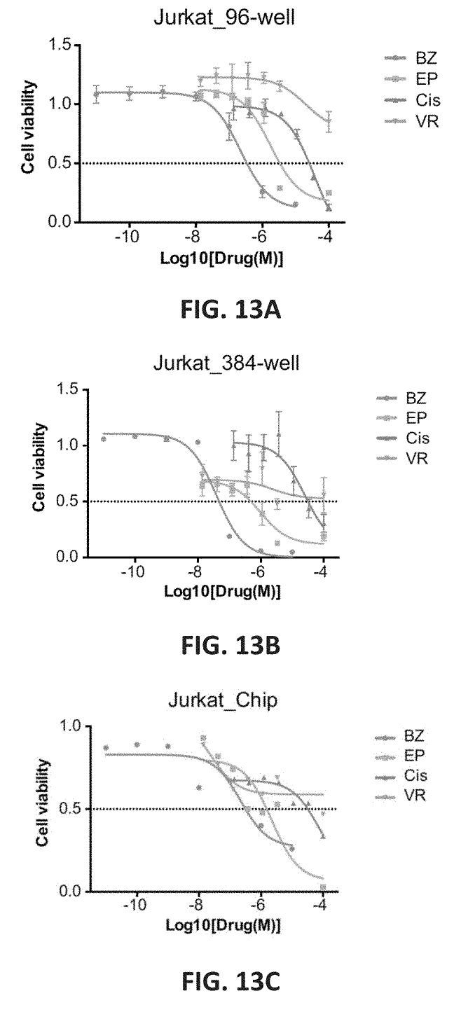

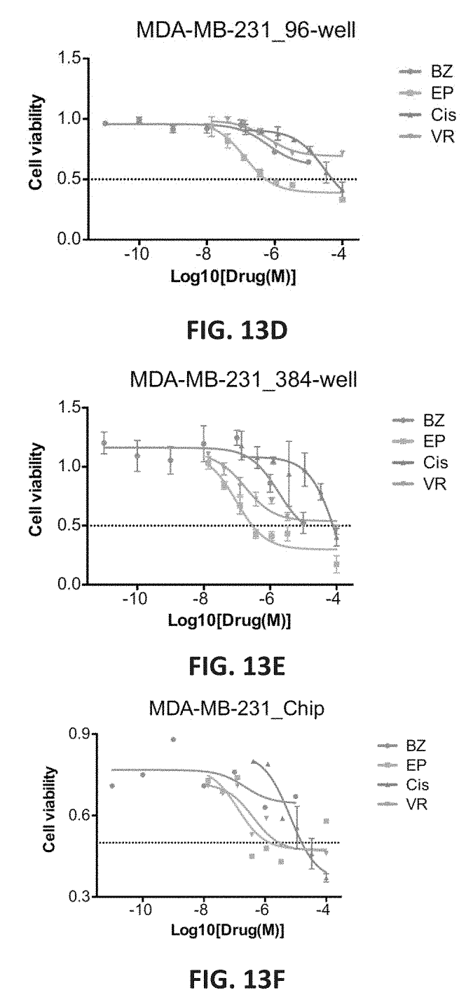

Next, we performed drug screening experiment on Jurkat cells and MDA-MB-231 cells, as models for suspended and adherent cells, respectively. We tested these two cell lines against four anti-cancer drugs, namely bortezomib, epirubicin, cisplatin and vorinostat (Table 1). Comparison of the chip screen results of both cell lines with those obtained from conventional plate reader assays using 96-well and 384-well microtiter plates manifested qualitative assessment of drug efficacy regardless of drug screening platform (FIG. 13), suggesting that our chip assay could be used to qualitatively assess whether a drug is effective or ineffective for the tumor tested in vitro. On the other hand, quantitative assessment of drug potency across the drug panel should be done on a single drug screening platform, in order to eliminate system errors and assay differences between platforms. Nevertheless, given that in vitro assays do not consider about pharmacokinetic differences between drugs in vivo, these results should be combined with clinical expertise to finalize therapeutic decision.

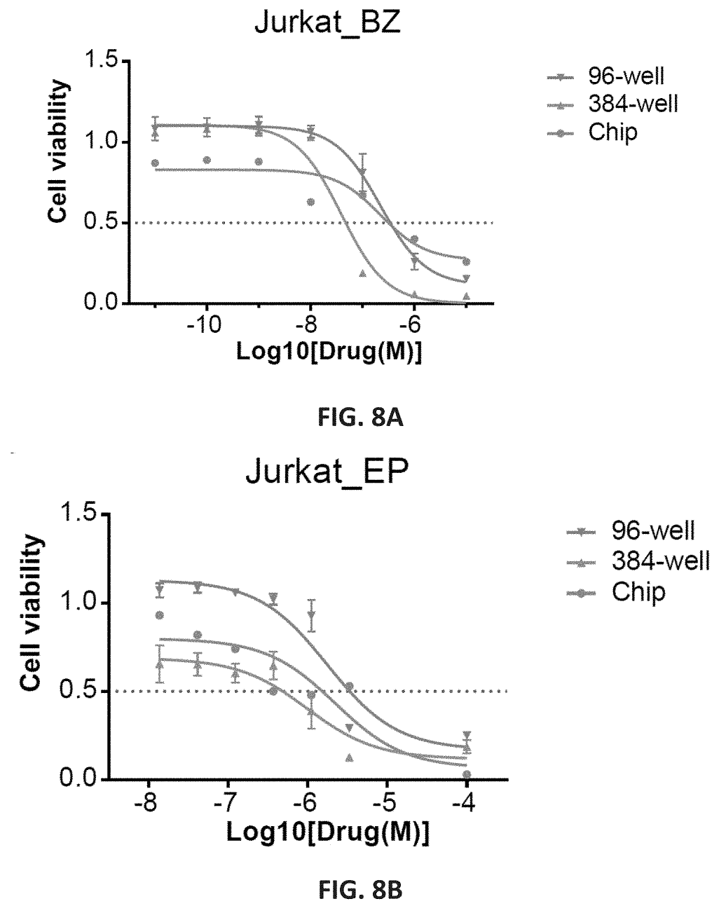

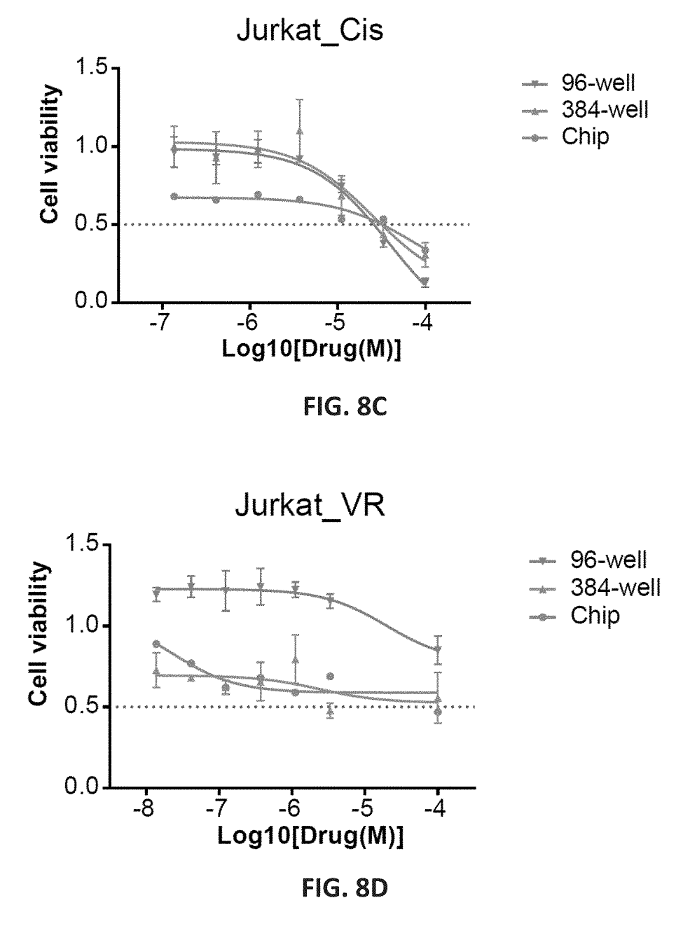

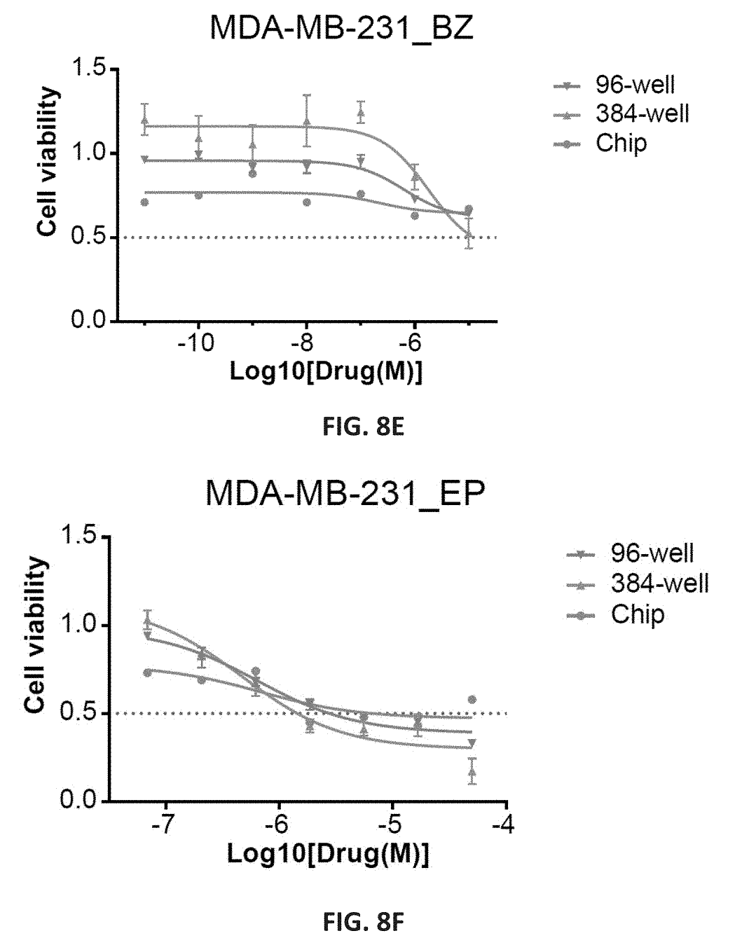

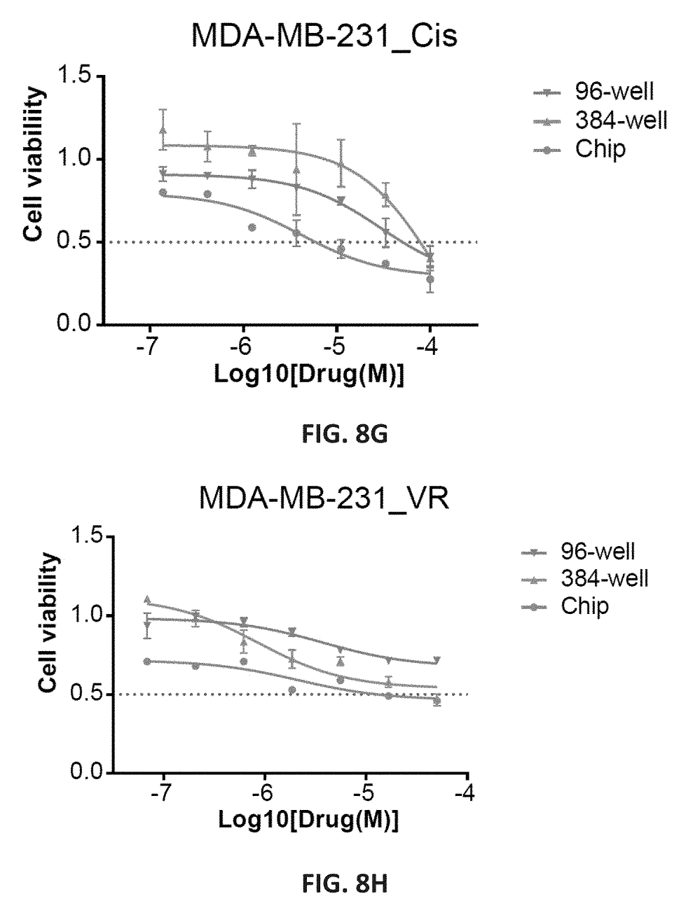

In this experiment, ranking of drugs under all three assays using 96-well plate (FIG. 13A), 384-well plate (FIG. 13B) and our microfluidic chip (FIG. 13C) were relatively consistent for Jurkat cells. In consideration of IC₅₀ values and lowest cell viability under highest drug dose tested, the ranking demonstrated that Jurkat cells were most susceptible to bortezomib, followed by epirubicin and then cisplatin, whereas vorinostat treatment showed a plateau above 50% cell viability under all doses tested in all three assays (FIGS. 13A-C). However, if we directly compared dose response curves across the screening platforms, there was less consistency between the curves (FIGS. 8A-D). For instance, in Jurkat cells treated with bortezomib, there was a prominent left shift of the dose response curve of 384-well plate screen (IC₅₀=39 nM) as compared to the 96-well plate screen data (IC₅₀=22.1 μM), whereas on chip screen data (IC₅₀=22.1 μM) prevailed higher conformity to 96-well late screen (FIG. 8A). Dose response curves of epirubicin treatment of Jurkat cells were more similar, with an IC₅₀ span of 1.0-2.2 μM on all three platforms (FIG. 8B). cisplatin treatment of Jurkat cells yielded almost identical response in Jurkat cells in both 96-well and 384-well plate assays (both IC₅₀=39.0 μM), whereas our chip assay gave identical IC₅₀ value of 39.0 μM but the dose response curve had smaller amplitude (FIG. 8C). Lastly, Jurkat cells were least susceptible to vorinostat treatment (all IC₅₀>50.0 μM), and this response was consistent in both plate reader assays and on chip (FIG. 8D). Taken together, our chip assay provided identical drug ranking assessment for Jurkat cells across the drug panel used in this study as compared to conventional plate reader assays.

Alternatively, MDA-MB-231 cells demonstrated less consistency in drug ranking among assays using 96-well plate (FIG. 13D), 384-well plate (FIG. 13E) and our microfluidic chip (FIG. 13F), epirubicin prevailed consistently lower IC₅₀ values towards MDA-MB-231 cells in all three assays, depicting IC₅₀ values of 0.6 μM and 0.5 μM for 96-well plate (FIG. 13D) and 384-well plate (FIG. 13E) assays respectively, whereas cell viability leveled off at approximately 50% on chip after reaching its IC₅₀ of 5.5 μM (FIG. 13F). The other three drugs exhibited more diverse effects on MDA-MB-231 cells among different platforms (FIGS. 13D-F). This trend was also observed in the lower curve conformity among the three assays under treatment of MDA-MB-231 cells with bortezomib (FIG. 8E), cisplatin (FIG. 8G) and vorinostat (FIG. 8H), as compared to epirubicin (FIG. 5F). For instance, MDA-MB-231 cells responded mildly to bortezomib, with IC₅₀ values of over 10.0 μM on chip and on 96-well plate, whereas 50% cell viability was observed at 10.0 μM bortezomib dose on 384-well plate (FIG. 8E). cisplatin showed IC50 values of over 50.0 μM in both 96-well and 384-well plates for MDA-MB-231 cells, whereas the cells showed 10-fold higher sensitivity towards this drug (IC₅₀=4.2 μM) on chip (FIG. 8G). vorinostat showed a plateau above 50% cell viability under 96-well plate and 384-well plate assays, while its IC₅₀ was 33.3 μM on chip (FIG. 8H). Taken together, there was discrepancy on drug ranking among all of the three drug screening platforms for MDA-MB-231 cells. Because MDA-MB-231 cells were cultured in adhesion on both 96-well and 384-well plates, and in suspension on chip, where the same culture medium recipe was used for all three assays carried out in parallel, we deduced that variant drug response was not attributed by culture condition and/or screening platform. Instead, we hypothesized that inherent cell heterogeneity of this cell line attributed to difference in drug response. Nevertheless, the most effective drug epirubicin was conserved in all three assays, suggesting that our chip was capable of indicating effective drug(s) from the drug panel even in heterogeneous cell population.

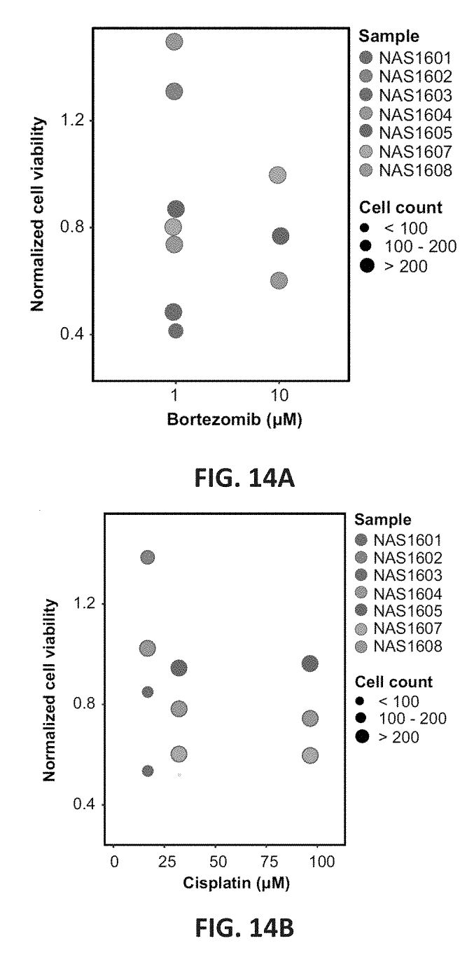

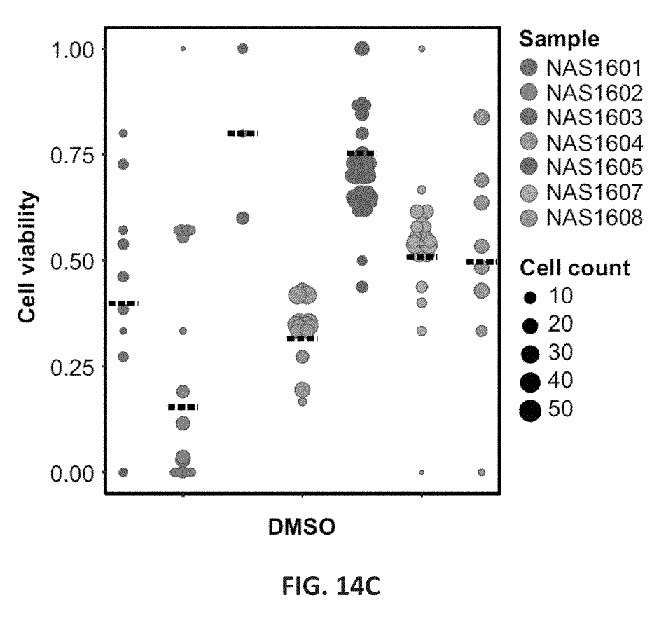

Close inspection of the dose response curves between different screening platforms prevailed overall smaller amplitude in the chip screen curves as compared to the plate reader assay curves (FIG. 5). This might be conferred by exploitation of single cell counting for cell viability assessment on chip, as compared to bulk population measurement of cellular metabolic activity on plate. In this aspect, our chip provided a preliminary tool to observe differential drug response in single cells, without compromising population analysis using multiple droplets for each drug treatment condition. Results showed that although cell viability differed vastly between individual droplets (FIGS. 14 and 15), analysis of the whole sample cell population in all droplets compensated for this variance and yielded reproducible results. For example, screening of MDA-MB-231 cells against cisplatin demonstrated comparable mean cell viability between two independent experiments, the standard deviation of which might be smaller than that of replicates in parallel experiments in 96-well or 384-well plates (FIG. 5G).

In conclusion, our chip assay provided qualitative drug potency assessment in MDA-MB-231 cells. It provided drug ranking assessment of Jurkat cells against the four anti-cancer drugs used in this study, which was comparable to that obtained from 96-well and 384-well plate reader assays.

### EXAMPLE 4

Drug Screening of Primary Tumor Dissociated Cells from Human Patients

Lastly, we screened seven primary nasopharyngeal tumors from human patients using our microfluidic chip assay. The tumors were collected by surgical resection from human cancer patients. The seven tumors varied in size from 0.2 cm to 0.5 cm in diameter and yielded variable cell numbers for use in this study from 0.5-1.0×10⁵ cells using identical tumor dissociation technique. Given the limited number of cells obtained from human primary tumors, parallel drug screening on 96-well or 384-well plates could be not be attained due to their requirement for higher cell numbers (Table 2). Consequently, all tumor samples were screened on chip against two anti-cancer drugs, namely bortezomib and cisplatin, and DMSO control for up to 5 conditions. Cells were treated for a total of 16-24 h and cell viability was measured by ethidium homodimer 1 staining.

TABLE 2
Comparison between different drag screening platforms.

| | 96-well plate | | 384-well plate | | PDMS chip | |
|---|---|---|---|---|---|---|
| Format | 8 × 12 | wells | 16 × 24 | wells | 2 channels of | |
| | | | | | 6 × 8 wells each | |
| Well bottom | Round, flat | | Square, flat | | N/A | |
| Cell growth area | 0.32 | cm2 | 0.06 | cm2 | N/A | |
| Culture medium per well | 50 | μL | 30 | μL | 21.39-26.91 | nL |
| Cells per well | 5.0 × 105 | cells | 1.0 × 105 | cells | 10-100 | cells |

| Cell density | 15.6 × 105 | cells/cm2 | 16.6 × 105 | cells/cm2 | N/A |
|---|---|---|---|---|---|
| Cell viability assay | Alamar Blue | | Alamar Blue | | Ethidium homodimer |
| | | | | | 1 staining |
| Viability assessment | Bulk population | | Bulk population | | Single cells |
| Measurement | Plate reader | | Plate reader | | Fluorescence microscope |
| Measurement time | 3 | minutes | 5 | minutes | 1 h (manual) 30 |
| | | | | | min (automatic) |
| Cell loading time | 1 h (manual) 10-20 min | | 1.5 h (manual) 20-30 | | 5 min (manual)? |
| | (robot) | | min (robot) | | (automatic) |
| Throughput | 8 drugs, 4-dose, | | 24 drugs, 4-dose, | | 5 drugs, 1-dose, single |
| | 3 replicates | | 4 replicates | | cell replicates |
| Cells per drug per dose | 1.5 × 106 | cells | 4.0 × 105 | cells | 16,000 cells (current) |
| | | | | | 100 cells (potential) |

| | | | |
|---|---|---|---|
| Cost per dose (without | HKD1.0 | HKD0.6 | HKD0.2 |
| drug) | | | |

Mock treatment of the seven human primary tumor cells showed diverse cellular response after 16-24 h treatment with DMSO alone (FIG. 14C). The range of cell viability among individual droplets within one sample also differed vastly, from 100% in the samples of NAS1602 and NAS1607 to the smallest range of 26.2% observed in NAS1604 sample (FIG. 14C). Of note, NAS1603 and NAS1605 exhibited over 70% mean cell viability, whereas the majority of primary tumor cells showed approximately 50% mean cell viability, and NAS1602 only retained 16.4% of viable cells after overnight incubation (FIG. 14C). Explanation of the diverse observed cell viability remained elusive.

Next, we compared the chip screen data of all seven human primary nasopharyngeal tumor samples against two anti-cancer drugs, namely bortezomib and cisplatin, respectively (FIGS. 14A and 14B). In order to illustrate difference in drug susceptibility among the seven human nasopharyngeal tumor samples, we normalized the drug treatment data of all samples to that of mock treatment to obtain normalized cell viability (FIGS. 14A and 14B). Results showed diverse response of the primary tumor cells towards the two drugs, with no explicit correlation to cellular response towards DMSO treatment in parallel experiments on the same chip (FIGS. 14A-C). Nevertheless, dose-dependent reduction of cell viability was observed in bortezomib treatment of two nasopharyngeal tumor samples, namely NAS1605 and NAS1608 (FIG. 14A). However, inverse correlation was observed in NAS1607 under bortezomib treatment (FIG. 14A), while its susceptibility leveled off under 100 μM and 33 μM cisplatin treatment (FIG. 14B). On the other hand, correlation between cisplatin concentration and cell viability was less prominent in all seven human primary tumor cells (FIG. 14B). Furthermore, we observed more fluctuations in normalized cell viability when it was close to 1.0, hypothesized to incur from subtle difference between single cells in the population. The low tumor amplification rate suggested of further optimization of our culture conditions for tumor amplification.

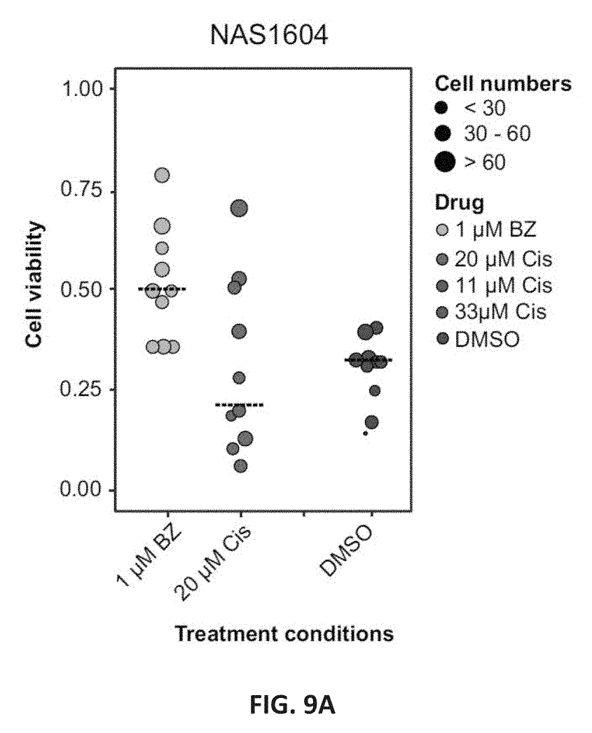

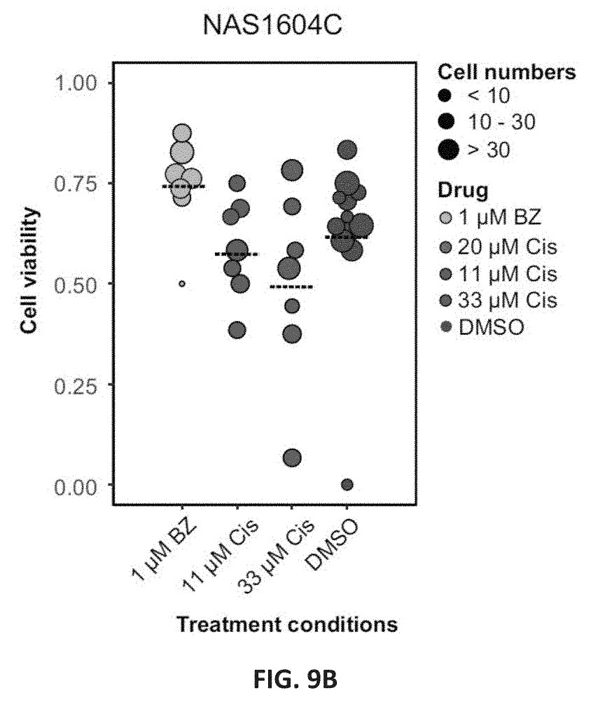

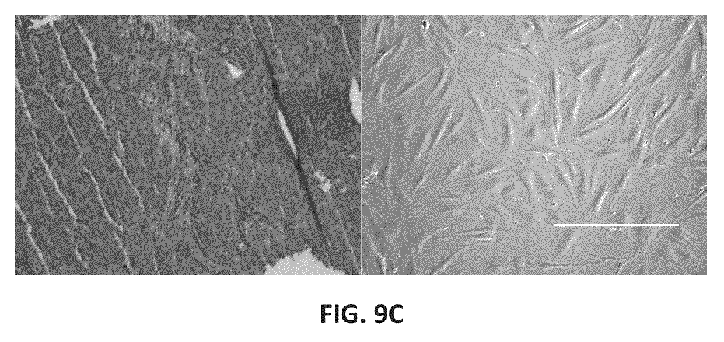

Drug susceptibility between the primary tumor cell NAS1604 and its derived cell line NAS1604C was compared (FIG. 9). NAS1604 primary tumor cells exhibited highest mean cell viability under 1 μM bortezomib treatment than DMSO treatment, whereas 20 μM cisplatin treatment exhibited lowest mean cell viability (FIG. 9A). Coincidently,the tumor cells amplified by 2D monolayer culture, named as NAS1604C, also exhibited highest cell viability under 1 μM bortezomib treatment as compared to mock treatment with DMSO, whereas dose-dependent response was observed in NAS1604C towards cisplatin (FIG. 9B). Revision of cellular drug response in individual droplets of NAS1604 demonstrated a larger range of droplet cell viability of 64.0% under cisplatin treatment, as compared to droplets treated with bortezomib (42.6%) or DMSO (26.2%) (FIG. 9A). Here, the concept of droplet cell viability was introduced to indicate the total number of live cells among all cells in each individual droplet; in turn, the range of droplet cell viability referred to the range of cell viability of all droplets under specified treatment condition. For instance, two droplets depicted 69.7% (n=44/66) and 5.7% (n=2/35) of maximum and minimum droplet cell viability, respectively, among all droplets under 20 μM cisplatin treatment in NAS1604 cells, resulting in 64.0% droplet cell viability range. In contrast to the primary tumor cell NAS1604, the droplet cell viability range among droplets treated with 1 μM bortezomib (37.5%) and 11 μM cisplatin (36.5%) in the derived cell line NAS1604C was smaller than corresponding conditions in the primary tumor cell NAS1604, whereas treatment of NAS1604C cells with 33 μM cisplatin (71.6%) or DMSO (83.3%) depicted larger droplet cell viability (FIG. 9B). However, close inspection of each treatment condition in the derived cell line NAS1604C showed one droplet outlier under the treatment conditions of 1 μM bortezomib, 33 μM cisplatin and DMSO (FIG. 9B), removal of which yielded droplet cell viability range of 16.1%, 40.8% and 25.0%, respectively, that were smaller than corresponding conditions in the primary tumor cell NAS1604. This observation suggested that the NAS1604 cells became more homogeneous during cell line derivatization. Indeed, the derived cells were fibroblast-like (FIG. 9C), whereas the Hematoxylin and Eosin (H&E) stained tumor tissue sections showed that the primary tumor cell NAS1604 was undifferentiated nasopharyngeal tumor (FIG. 9C), indicating that cell morphology changed during cell derivatization Nonetheless, the molecular mechanism remained obscure. On the other hand, mean cell viability of all treatment conditions were higher in the derived cell line NAS1604C than corresponding conditions in the primary tumor cell NAS1604, reminiscent of increased cell viability that was required for tumor amplification.

Collectively, these data reflected that: (1) primary tumor cells had diverse susceptibility towards different drugs, thus supporting the need for personalized cancer therapy; and (2) primary tumor cells before and after in vitro amplification (primary tumor cells are re-named as primary tumor cell lines after in vitro amplification in the present disclosure) might prevail similar drug susceptibility, while their morphological cell type might be different.

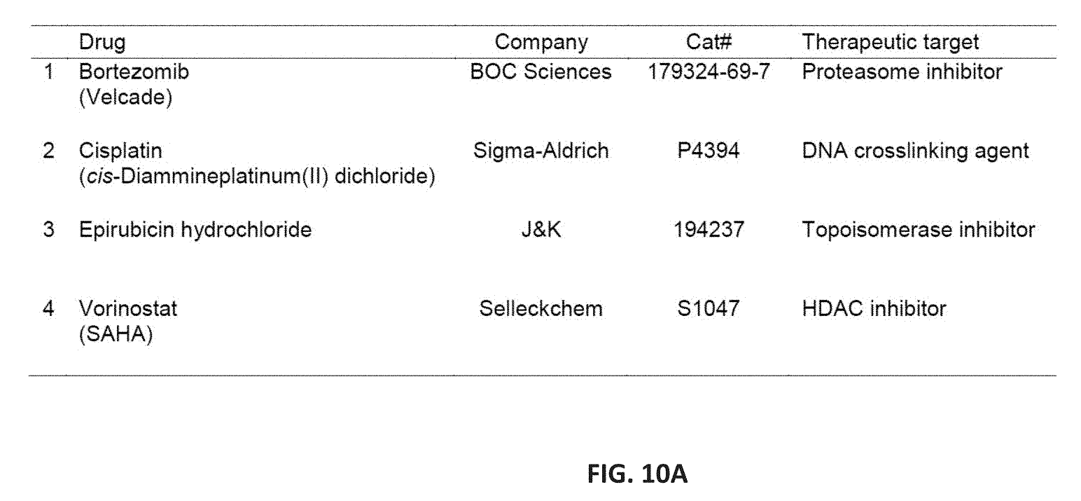

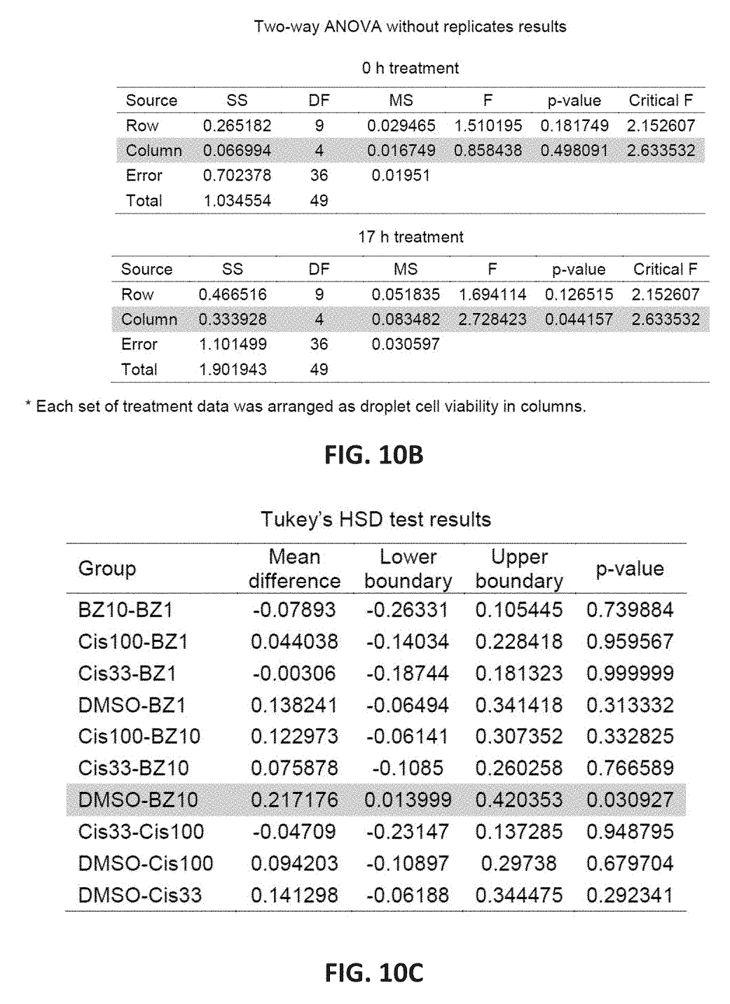

In order to exemplify the capability of our microfluidic chip, one nasopharyngeal tumor sample was shown in details as an example (FIG. 15). First, cell imaging immediately after chip loading showed a certain extent of ethidium homodimer 1-labelled cells (FIG. 15A), which was roughly consistent to Trypan Blue staining results of 37% measured by cell counter (data not shown). Cell viability measurement by ethidium homodimer 1 staining at 0 h showed no significant difference in mean cell viability among different treatment conditions (FIG. 15B). However, mean cell viability of NAS1608 cells declined from 75.8% to 56.3% before and after 17 h DMSO treatment (FIGS. 15B and 15C). Decrease in mean cell viability from 78.8% to 34.6% and from 72.9% to 42.5% was observed after 17 h of 10 μM and 1 μM bortezomib treatment respectively (FIGS. 15B and 15C). cisplatin treatment led to a reduction of mean cell viability of 23.4% and 27.7% under the concentrations of 100 μM and 33 μM respectively (FIGS. 15B and 15C). Normalization to DMSO-treated negative control (n=264) and calibration by 0 h treatment data showed that 17 h treatment of NAS1608 cells with 10 μM bortezomib resulted in 51.7% mean cell death (n=582), while 100 μM cisplatin led to 21.0% mean cell death (n=427). Statistical analysis of the drug response of NAS1608 cells towards bortezomib and cisplatin showed significant difference in two-way ANOVA, whereas Tukey's honest significant difference (HSD) test confirmed the difference between 10 μM bortezomib and DMSO treatment for NAS1608 human primary nasopharyngeal tumor sample (Table 2). FIGS. 10B and 10C show the statistical analysis of all five data sets of NAS1608 human primary nasopharyngeal tumor sample treated with 10 μM Bortezomid (BZ10), 1 μM bortezomib (BZI), 100 μM cisplatin (Cis100), 33.3 μM cisplatin (Cis33) and DMSO (DMSO) at 0 h and 17 h post-treatment. First, two-way ANOVA was performed in Microsoft Excel. Next, post hoc analysis using Turkey's honest significant difference (HSD) test was performed in R v. 3.3.2. Significant difference was highlighted din yellow.

Further investigation into drug response among individual droplets showed stark contrast before and after overnight incubation (FIGS. 15B and 15C). Before treatment, the range of droplet cell viability was 29.0%, 31.1% and 16.2% under treatment with 10 μM bortezomib, 100 μM cisplatin and DMSO, respectively (FIG. 15B). 17 h post-treatment demonstrated a droplet cell viability range of 49.7%, 56.5% and 50.5% under treatment with 10 μM bortezomib, 100 μM cisplatin and DMSO, respectively (FIG. 15C), Thus, difference in the range of droplet cell viability before and after DMSO treatment was largest (34.2%), suggesting that sonic cells might have responded to mock treatment while others did not. To dissect this phenomenon, we looked at individual droplets before and after treatment. In 5 out of 8 wells (n=207), DMSO induced less than 20% decrease in droplet cell viability, whereas 1 well (n=53) showed 27.0% decrease in droplet cell viability, and 2 wells (n=57) showed over 30% decrease in droplet cell viability. Hence, droplets that exhibited droplet cell viability reduction of over 30% under DMSO treatment contained merely 21.6% of the total sample cell population (n=57/264). In contrast, 10 μM bortezomib treatment reduced droplet cell viability by a minimum of 29.3% (n=44), and a maximum of 62.9% (n=74), whereas the majority of droplets exhibited a droplet cell viability reduction of 35-55% (n=464). Hence, droplets that prevailed droplet cell viability reduction of over 30% under 10 μM bortezomib contained 92.4% of the total sample cell population (n=538/582). Taken together, our data suggested that NAS1608 cells responded to 10 μM bortezomib with higher cell numbers and conformity as compared to DMSO, thus giving significant difference to bortezomib treatment as compared to DMSO treatment.

In conclusion, our data demonstrated that mean cell viability could be used to reveal the percentage of cells that responded to the investigated drug(s), applied to qualitative drug potency assessment and drug ranking. On the other hand, the range of droplet cell viability suggested the conformity of cellular drug response towards the investigated drug(s).

Discussion

In this study, we used a centimeter-sized PDMS-based droplet microfluidic chip to provide efficient evaluation of drug susceptibility of cancers. Our data indicated that our system could be used to screen as few as 16,000 cells obtained from primary cancer for each treatment condition within 2.4 h after tumor resection from cancer patients. Rapid screening for effective therapy is virtuous, especially for fast-growing cancers from the pancreas (20.8%), lung (32.1%), brain (40.1%) and oesophagus (41.9%), which kills patients within one year after diagnosis. Our current assay provided evidence for rapid drug potency assessment within 2.4 h. This would allow clinical doctors to determine their patient's therapeutic regime within 2 days. Furthermore, the cost of our chip was merely HKD0.20 per chip (Table 2), making it pragmatically affordable for all cancer patients. Hence, our technology provided unprecedented opportunity for rapid evidence-based decision making for personalized cancer therapy.

Our microfluidic chip was designed so that the loading of samples can be performed by injecting a sequence of samples segregated by a carrier fluid, i.e. oil phase, through an inlet of the droplet-forming channel, which allows high throughput screening that otherwise cannot be realized by existing microfluidic devices using pipette loading methods. More importantly, cell lines were prone to survive and proliferate on chip, whereas primary tumor cells died quickly under mock treatment after 16-2.4 h treatment (FIG. 14C). Therefore, the capability of quick loading and drug screening of the microfluidic chip of the present disclosure makes the chip versatile for different cells even for primary cells that are vulnerable and have limited amount.

Ethidium homodimer 1 staining was used to measure single cell viability in this assay. Our data supported for observation of differential drug response in cancer cell lines and primary tumor cells from human cancer patients. On the population scale, mean cell viability obtained on chip could be applied to assessing drug potency and ranking drugs in a drug panel. Additionally, the conformity of drug response could be implied from the range of droplet cell viability.

In conclusion, our microfluidic chip assay provides a powerful tool for rapid, low-input drug screening of primary cancers. Adaptation of the assay to suspended and adherent cancer cell lines suggests of its application in potentially all types of cancers. It provides us the opportunity to observe and quantify cellular drug response on the single cell level, whereas population analysis is achievable by statistical analysis of multiple droplets.

Although the invention has been described in terms of certain embodiments, other embodiments apparent to those of ordinary skill in the art are also within the scope of this invention. Accordingly, the scope of the invention is intended to be defined only by the claims which follow.

1. A microfluidic chip (100) for generating a plurality of droplets from a loading fluid, comprising at least one droplet-forming channel (200), each of the at least one droplet-forming channel (200) comprising:
plural droplet-forming units serially connected together;
an inlet (201) for receiving the loading fluid and providing the loading fluid to the plural droplet-forming units; and
an outlet (202) for discharging the loading fluid remained after passing through the plural droplet-forming units;
wherein:
an individual droplet-forming unit (209) comprising an inflow channel (203), a neck channel (204), a droplet-forming well (205), a restricted flow port element (206), and an outflow channel (207) all of which are sequentially arranged along a flow direction of the loading fluid;
the inflow channel (203) is configured to accept the loading fluid and is in fluid communication with the neck channel (204),
the neck channel (204) is in fluid communication with the droplet-forming well (205) for delivering a first portion of the loading fluid from the inflow channel (203) to the droplet-forming well (205), and is configured to have a cross-sectional width that is smaller than a cross-sectional width of the droplet-forming well (205) to prevent droplet escape from the droplet-forming well (205);
the restricted flow port element (206) is configured to generate a restricted flow to facilitate droplet formation in the droplet-forming well (205); and
wherein:
the individual droplet-forming unit (209) further comprises a bypass channel (208);
the bypass channel (208) is located around the droplet-forming well (205), and is configured to deliver a second portion of the loading fluid from the inflow channel (203) to the outflow channel (207).

2. The microfluidic chip (100) of claim 1, wherein the neck channel (204) and the bypass channel (208) have a cross-sectional width ratio of the bypass channel to the neck channel, the cross-sectional width ratio being selected such that the first portion of the loading fluid fills the droplet-forming well (205) before the second portion of the loading fluid fills the bypass channel (208).

3. The microfluidic chip (100) of claim 2, wherein the cross-sectional width ratio of the bypass channel to the neck channel is approximately 0.2 to approximately 1.0.

4. The microfluidic chip (100) of claim 2, wherein the cross-sectional width ratio of the bypass channel to the neck channel is approximately 0.75.

5. The microfluidic chip (100) of claim 1, wherein the neck channel (204) has a cross-sectional width of approximately 50-150 μm.

6. The microfluidic chip (100) of claim 1, wherein the droplet-forming well (205) has a cross-sectional width of approximately 100-500 μm.

7. The microfluidic chip (100) of claim 1, wherein the restricted flow port element (206) is a restriction channel having a cross-sectional width of approximately 5-20 μm.

8. A mold comprising complementary features to a microfluidic chip (100), the microfluidic chip (100) comprising:
at least one droplet-forming channel (200), each of the at least one droplet-forming channel (200) comprising:
plural droplet-forming units serially connected together;
an inlet (201) for receiving the loading fluid and providing the loading fluid to the plural droplet-forming units; and
an outlet (202) for discharging the loading fluid remained after passing through the plural droplet-forming units;
wherein:
an individual droplet-forming unit (209) comprises an inflow channel (203), a neck channel (204), a droplet-forming well (205), a restricted flow port element (206), and an outflow channel (207) all of which are sequentially arranged along a flow direction of the loading fluid;
the inflow channel (203) is configured to accept the loading fluid and is in fluid communication with the neck channel (204);
the neck channel (204) is in fluid communication with the droplet-forming well (205) for delivering a first portion of the loading fluid from the inflow channel (203) to the droplet-forming well (205), and is configured to have a cross-sectional width that is smaller than a cross-sectional width of the droplet-forming well (205) to prevent droplet escape from the droplet-forming well (205);
the restricted flow port element (206) is configured to generate a restricted flow to facilitate droplet formation in the droplet-forming well (205); and
wherein:
the individual droplet-forming unit (209) further comprises a bypass channel (208);
the bypass channel (208) is located around the droplet-forming well (205), and is configured to deliver a second portion of the loading fluid from the inflow channel (203) to the outflow channel (207).

9. The mold of claim 8, wherein the mold is made of a material selected from the group consisting of crystalline silicon, amorphous silicon, glass, quartz, and metals.

10. The mold of claim 8, wherein the neck channel (204) and the bypass channel (208) have a cross-sectional width ratio of the bypass channel to the neck channel, the cross-sectional width ratio being selected such that the first portion of the loading fluid fills the droplet-forming well (205) before the second portion of the loading fluid fills the bypass channel (208).

11. The mold of claim 8, wherein the cross-sectional width ratio of the bypass channel to the neck channel is approximately 0.75.

12. A method for drug screening, wherein the method comprising steps of:
f) providing the microfluidic chip (100) of claim 1;
g) flushing the droplet-forming channel (200) with a carrier fluid from the outlet 202 to the inlet (201);
h) infusing a loading fluid comprising of a sample fluid and a carrier fluid in distinct layers separated by an interface from the inlet (201) into the droplet-forming channel (200) to form droplets comprising the sample fluid;
i) sealing the inlet (201) and the outlet (202); and
j) imaging the droplets comprising the sample fluid.

13. The method of claim 12, wherein the carrier fluid comprises an oil and a surfactant.

14. The method of claim 13, wherein the carrier fluid is a perfluorinated trialkyl amine oil supplemented with approximately 1-5% fluorosurfactant.

15. The method of claim 12, wherein the sample fluid comprises cells, a drug, a cell culture medium, an additive, a dead cell indicator, and/or a metabolic indicator.

16. The method of claim 15, wherein the cells are cancer cells selected from the group consisting of cancer cell lines, primary tumor cells, secondary tumor cells, cancer stem cells, and circulating tumor cells.

17. The method of claim 15, wherein the cell culture medium comprises fetal bovine serum at a concentration of 1%-20% (v/v).

18. The method of claim 15, wherein the additive is methyl cellulose.

19. The method of claim 15, wherein the dead cell indicator is selected from the group consisting of ethidium homodimer 1, Alamar Blue, SYTOX Green nucleic acid stain, and propidium iodide; and the metabolic indicator is selected from the group consisting of Calcein AM, C₁₂-resazurin, SYTO 10 dye, and SYBR 14 nucleic acid stain.

20. The method of claim 18, wherein the methyl cellulose has a percentage of 0.5%-3% (m/v) in the sample fluid.
